(19) United States
(12) Patent Application Publication
(10) Pub. No.: US 2009/0012130 A1
(43) Pub. Date: Jan. 8, 2009

(54) Strategies for Designing Drugs that Target the Sir2 Family of Enzymes

(75) Inventors: Cynthia Wolberger, Baltimore, MD (US); Jose L. Avalos, Waltham, MA (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, BALTIMORE, MD (US)

(21) Appl. No.: 11/883,015

(22) PCT Filed: Jan. 25, 2006
(86) PCT No.: PCT/US2006/002713

§ 371 Date: Aug. 28, 2008

Related U.S. Application Data
(60) Provisional application No. 60/646,792, filed on Jan. 25, 2005.

(51) Int. Cl. A61K 31/4406; C12Q 1/34; A61P 35/00

(52) U.S. Cl. 514/355; 435/18

(57) ABSTRACT

The instant invention describes methods of identifying compounds that modulate the activity of Sir2 enzymes. Sir2 enzymes form a unique class Of NAD⁺ dependent deacetylases required for diverse biological processes including transcriptional silencing, regulation of apoptosis, fat mobilization, and lifespan regulation. Sir2 activity is regulated by nicotinamide, a non-competitive inhibitor that promotes a base exchange reaction at the expense of deacetylation.

### RELATED APPLICATION

This application claims the benefit of U.S. provisional patent application Ser. No. 60/646,792, filed Jan. 25, 2005, the disclosure of which is incorporated herein in its entirety by this reference.

### BACKGROUND

Sir2 enzymes, also known as sirtuins, comprise an ancient family of NAD⁺-dependent deacetylases (Imai et al., 2000, Nature 403, 795-800; Landry et al., 2000, Proc Natl Acad Sci USA 97, 5807-5811; Smith et al., 2000, Proc Natl Acad Sci USA 97, 6658-6663) that are conserved from bacteria to humans and play a role in a wide variety of important biological processes, including transcriptional silencing (Brachmann et al., 1995, Genes Dev 9, 2888-2902), DNA recombination (Gottlieb and Esposito, 1989, Cell 56, 771-776; McMurray and Gottschling, 2003, Science 301, 1908-1911) and repair (Bennett et al., 2001, Mol Cell Biol 21, 5359-5373), apoptosis (Brunet et al., 2004, Science 303, 2011-2015; Luo et al., 2001; Vaziri et al., 2001, Cell 107, 149-159), axonal protection (Araki et al., 2004, Science 305, 1010-1013), fat mobilization (Picard et al., 2004, Nature 429, 771-776) and aging (Kaeberlein et al., 1999, Genes Dev 13, 2570-2580; Lin et al., 2000, Science 289, 2126-2128). Sirtuins are highly conserved and contain a conserved catalytic domain of approximately 275 amino acids (Grozinger, C. M. et al., 2001, J. Biol. Chem. 276, 38837-38843). In humans, eight homologues have been identified.

Overexpression or hyper-activation of Sir2 enzymes increases lifespan in yeast (Kaeberlein et al., 1999, Genes Dev 13, 2570-2580), worms (Tissenbaum and Guarente, 2001, Nature 410, 227-230) and flies (Wood et al., 2004, Nature 430, 686-689) while deletion or inhibition of sirtuins shortens lifespan (Kaeberlein et al., 1999, Genes Dev 13, 2570-2580). Consistent with their diverse roles in biology, a variety of proteins are deacetylated by sirtuins, including histones (Imai et al., 2000, Nature 403, 795-800), acetyl-coA synthetase (Starai et al., 2002, Science 298, 2390-2392), α-tubulin (North et al., 2003, Mol Cell 11, 437-444), myoD (Fulco et al., 2003, Mol Cell 12, 51-62), p53 (Luo et al., 2001, Cell 107, 137-148; Vaziri et al., 2001, Cell 107, 149-159), Foxo3 (Brunet et al., 2004, Science 303, 2011-2015; Motta et al., 2004, Cell 116, 551-563), Ku70 (Cohen et al., 2004) and NF-κB (Yeung et al., 2004, Embo J 23, 2369-2380). Sirtuins deacetylate lysine residues in an unusual chemical reaction that allows them to be tightly regulated in the cell. The deacetylation reaction catalyzed by these enzymes is coupled to the cleavage of NAD⁺, yielding nicotinamide and O-acetyl ADP-ribose (OAADPr), along with the deacetylated lysine (Denu, 2003, Trends Biochem Sci 28, 41-48; Sauve et al., 2001, Biochemistry 40, 15456-15463; Sauve and Schramm, 2004, Curr Med Chem 11, 807-826). The nicotinamide product is a non-competitive inhibitor of sirtuins (Bitterman et al., 2002, J Biol Chem 277, 45099-45107), thereby allowing theses enzymes to be modulated by nicotinamide levels in the cell, as well as by NAD⁺.

The Sir2 protein is a deacetylase which uses NAD as a cofactor (Imai et al., 2000, Nature, 403:795-800; Smith et al., 2000, Proc. Natl. Acad. Sci. USA, 97:6658-6663; Tanner et al., 2000, Proc. Natl. Acad. Sci. USA, 97:14178-14182; Tanny and Moazed, 2001, Proc. Natl. Acad. Sci. USA, 98:415-420). Unlike other deacetylases, many of which are involved in gene silencing, Sir2 is insensitive to histone deacetylase inhibitors like trichostatin A (TSA) (Imai et al., 2000, Nature, 403:795-800; Landry et al., 2000, Biochem. Biophys. Res. Commun., 278:685-690; Smith et al., 2000, Proc. Natl. Acad. Sci. USA, 97:6658-6663). Deacetylation of acetyl-lysine by Sir2 is tightly coupled to NAD hydrolysis, producing nicotinamide and a novel acetyl-ADP ribose compound (1-O-acetyl-ADP-ribose) (Tanner et al., 2000, Proc. Natl. Acad. Sci. USA, 97:14178-14182; Landry et al., 2000, Proc. Natl. Acad. Sci. USA, 97:5807-5811; Tanny and Moazed, 2001, Proc. Natl. Acad. Sci. USA, 98:415-420). The NAD-dependent deacetylase activity of Sir2 is essential for its functions which can connect its biological role with cellular metabolism in yeast (Guarente, 2000, Genes Dev., 14:1021-1026; Lin et al., 2000, Science, 289:2126-2128; Smith et al., 2000, Proc. Natl. Acad. Sci. USA, 97:6658-6663). Mammalian Sir2 homologs have NAD-dependent histone deacetylase activity. Most information about Sir2 mediated functions comes from the studies in yeast.

In yeast, these proteins form complexes with other proteins to silence chromatin by accessing histones and deacetylating them (Moretti, P., et al. (1994) Genes Dev. 8, 2257-69; Rine, J., and Herskowitz, I. (1987) Genetics 116, 9-22; Shou, W., et al. (1999) Cell 97, 233-44; Straight, A. F., et al. (1999) Cell 97, 245-56; Hecht, A., et al. (1995) Cell 80, 583-92; Johnson, L. M., et al. (1990) Proc. Natl. Acad. Sci. U.S.A. 87, 6286-90; Braunstein, M., et al. (1993) Genes Dev. 7, 592-604; Imai, S., et al. (2000) Nature 403, 795-800; Landry, J., et al. (2000) Proc. Natl. Acad. Sci. U.S.A. 97, 5807-11; Smith, J. S., et al. (2000) Proc. Natl. Acad. Sci. U.S.A. 97, 6658-63). Sir2 enzymes are homologs of the bacterial enzyme cobB, a phosphoribosyl transferase (Tsang, A. W., and Escalante-Semerena, J. C. (1998) J. Biol. Chem. 273, 31788-94), which led to the finding that Sir2p employs NAD⁺ as a co-substrate in deacetylation reactions (Landry, J., Slama, J. T., and Sternglanz, R. (2000) Biochem. Biophys. Res. Commun. 278, 685-90; Imai, S., et al. (2000) Nature 403, 795-800; Smith, J. S., et al. (2000) Proc. Natl. Acad. Sci. U.S.A. 97, 6658-63). This unusual requirement for NAD⁺ is stoichiometric and generates a novel product originally proposed to be β-1′-AADPR (Tanner, K. G., et al. (2000) Proc. Natl. Acad. Sci. U.S.A. 97, 14178-82; Tanny, J. C., and Moazed, D. (2001) Proc. Natl. Acad. Sci. U.S.A. 98, 415-20) or possibly 2′-AADPR (Moazed, D. (2001) Curr. Opin. Cell Biol. 13, 232-8).

The distribution of Sir2p family of enzymes into organisms without histone substrates, and eukaryotic genomes encoding multiple Sir2 proteins, suggest a family of deacetylases with varying substrates. Mutagenesis experiments suggest that the N- and C-terminal regions flanking the catalytic core domain of Sir2p help direct it to different targets (Cuperus, G., et al. (2000) Embo. J. 19, 2641-51).

Lysine deacetylation by sirtuins, however, extends beyond histones. Targets of sirtuin regulatory deacetylation include mammalian transcription factors such as p53 (Luo, J. et al. (2001) Cell 107, 137-48; Vaziri, H. et al. (2001) Cell 107, 149-59; Langley E. et al. (2002) EMBO J. 21, 2383-2396), the cytoskeletal protein, tubulin (North, B. J. et al. (2003) Molecular Cell 11, 437-444) and the bacterial enzyme, acetyl-CoA synthetase (Starai, V. J. et al. (2002) Science 298, 2390-2392; Zhao, K. et al. (2004) J. Mol. Biol. 337, 731-741).

Recently, a great deal of insight has been gained into the biochemistry of Sir2-like deacetylases (Vinitsky, A., et al. (1991) J Bacteriol 173(2), 536-40). In vitro, Sir2 has specificity for lysine 16 of histone H4 and lysines 9 and 14 of histone H3 (Kennedy, B. K., et al. (1994) J Cell Biol 127(6 Pt 2), 1985-93; Ashrafi, K., et al. (2000) Genes Dev 14(15), 1872-85). Although TSA sensitive HDACs catalyze deacetylation without the need of a cofactor, the Sir2 reaction requires NAD+. This allows for regulation of Sir2 activity through changes in availability of this co-substrate (Kennedy, B. K., et al. (1994) J Cell Biol 127(6 Pt 2), 1985-93; Kim, S., et al. (1996) Biochem Biophys Res Commun 219(2), 370-6; Ashrafi, K., et al. (2000) Genes Dev 14(15), 1872-85). Sir2 deacetylation is coupled to cleavage of the high-energy glycosidic bond that joins the ADP-ribose moiety of NAD+ to nicotinamide. Upon cleavage, Sir2 catalyzes the transfer of an acetyl group to ADP-ribose (Kennedy, B. K., et al. (1994) J Cell Biol 127(6 Pt 2), 1985-93; Kim, S., et al. (1996) Biochem Biophys Res Commun 219(2), 370-6; Jazwinski, S. M. (2001) Mech Ageing Dev 122(9), 865-82; Imsande, J. (1964) Biochim. Biophys. Acta 85, 255-273). The product of this transfer reaction is O-acetyl-ADP-ribose.

Recently, several groups (Luo, J. et al. (2001) Cell 107, 137-48; and Vaziri, H. et al. (2001) Cell 107, 149-59) have explored the influence of the mammalian homologues, Sir2α (the mouse homologue of S. cerevisiae Sir2, also known as mSIRT1) and Sir2α (the human homologue of S. cerevisiae Sir2, also known as hSIRT1), on the activity of the p53 tumor suppressor gene. These studies indicate that deacetylase activity of Sir2α and SIR2α act on p53, resulting in suppression of the tumor suppressor activity. They have also shown that this deacetylase activity is dependent on nicotinamide adenosine dinucleotide (NAD).

The instant invention provides a method for identifying compounds that are agonists or inhibitors of the NAD+ dependent deacetylase activity of a member of the Sir2 family of proteins using a small molecule, wherein the small molecule binds to the C region or the flexible loop of the enzyme.

### BRIEF SUMMARY OF THE INVENTION

In one aspect, the invention provides a method for identifying a compound which modulates the activity of a Sir2 enzyme, the method comprising: a) contacting a Sir2 enzyme with a compound under conditions suitable for modulation of the activity of the Sir2 enzyme; and b) detecting modulation of the activity of the Sir2 enzyme by the compound; wherein the compound is capable of interacting with the C-pocket of the Sir2 enzymes.

In another aspect, the invention provides a method for identifying a compound which modulates the binding of a Sir2 enzyme, the method comprising: a) contacting a Sir2 enzyme with a compound under conditions suitable for modulation of the binding of the Sir2 enzyme; and b) detecting modulation of the binding of the Sir2 enzyme by the compound; wherein the compound is capable of interacting with the C-pocket of the Sir2 enzymes.

In still another aspect, the invention provides a method of modulating the Sir2 activity in a subject, the method comprising administering to the subject a compound identified by: a) contacting a Sir2 enzyme with a compound under conditions suitable for modulation of the activity of the Sir2 enzyme; and b) detecting modulation of the activity of the Sir2 enzyme by the compound; wherein the compound is capable of interacting with the C-pocket of the Sir2 enzymes.

In yet another aspect, the invention provides a method for identifying a compound which modulates the activity of a Sir2 enzyme, the method comprising: a) contacting a Sir2 enzyme with a compound under conditions suitable for modulation of the activity of the Sir2 enzyme; and b) detecting modulation of the activity of the Sir2 enzyme by the compound; wherein the compound interacts with the flexible loop of the Sir2 enzyme.

In another aspect, the invention provides a method for identifying a compound which modulates the binding of Sir2 enzymes, the method comprising: a) contacting a Sir2 enzyme with a compound under conditions suitable for modulation of the binding of the Sir2 enzyme; and b) detecting modulation of the binding of the Sir2 enzyme by the compound; wherein the compound is interacts with the flexible loop of the Sir2 enzyme.

In still another aspect, the invention provides a method of modulating the Sir2 activity in a subject, the method comprising administering to the subject a compound identified by: a) contacting a Sir2 enzyme with a compound under conditions suitable for binding or modulation of the activity of the Sir2 enzyme; and b) detecting modulation of the activity or the binding of the Sir2 enzyme by the compound; wherein the compound is interacts with the flexible loop of the Sir2 enzyme.

In another aspect, the invention provides a method for identifying a compound which modulates the activity of Sir2 enzymes, the method comprising: a) contacting a Sir2 enzyme, attached to a fluorophore and a fluorescence quencher, with a test compound at 37° C.; and

b) detecting the fluorescence of the Sir2 enzyme by the test compound at 340 nm; wherein the test compound binds to the flexible loop region of the Sir2 enzymes.

In another aspect, the invention provides a method for identifying a compound which modulates the binding or activity of Sir2 enzymes, the method comprising: a) creating a computer model of the structure of the flexible loop of a Sir2 enzyme based on the crystal structure of the Sir2 enzyme; b) introducing a compound to the flexible loop region of the Sir2 enzyme; and c) determining from computer calculations whether the compound interacts with the flexible loop region of the Sir2 enzyme.

In another aspect, the invention provides a method for identifying a compound which modulates the binding or activity of Sir2 enzymes, the method comprising: a) creating a computer model of the structure of the flexible loop of a Sir2 enzyme based on the three-dimensional structure coordinates of any of FIG. 8; Table 1, of the Sir2 enzyme; b) introducing a compound to the flexible loop region of the Sir2 enzyme; and c) determining from computer calculations whether the compound interacts with the flexible loop region of the Sir2 enzyme.

In other aspects, the invention further comprises the intention of identifying compounds that bind to the flexible loop.

In another aspect, the invention provides a method of treating a disorder in a subject, comprising administering to said subject in need thereof, an effective amount of a compound identified, such that said subject is treated for said disorder.

### DETAILED DESCRIPTION

### Brief Description of the Drawings

FIG. 1: Overview of the mechanism of the sirtuin-catalyzed NAD⁺-dependent deacetylation and nicotinamide regulation.

(A) The initial step of catalysis involves a nucleophilic attack of the carbonyl oxygen of acetyl-lysine on the C1′ of the N-ribose of NAD⁺. This step forms an O-alkylamidate intermediate that is consumed by the internal attack of its 2′OH, activated by a conserved histidine, leading to deacetylation, or by the attack of a nicotinamide molecule on the β-face of its C1′, which leads to nicotinamide exchange and inhibition of deacetylation.

(B) In the absence of substrate peptide, NAD⁺ can bind in the A and B pockets of sirtuins in alternative, non-productive conformations.

(C) In the presence of a substrate peptide, NAD⁺ binds in a precise productive conformation that buries its nicotinamide moiety in the highly conserved C pocket of sirtuins.

FIG. 2. Crystal structures of Sir2Af2 and Sir2Tm bound to nicotinamide.

(A) Structure I: Sir2Af2 bound to nicotinamide and non-productive NAD⁺ with a highlighted flexible loop.

(B) Structure II: Sir2Af2 bound to nicotinamide and ADP-ribose, flexible loop highlighted.

(C) Structure III: Sir2Tn1 bound to nicotinamide and acetylated p53 peptide, flexible loop highlighted.

(D) Superposition of the different conformations of the flexible loop observed in the known structures of Sir2Af2.

(E-G) Simulated annealing omit maps showing nicotinamide density. 2Fo-Fc map is contoured at 1σ and the Fo-Fc map at 3σ. (E) Structure I, (F) Structure II, (G) Structure III.

FIG. 3. Surface representation of the sirtuin active site pockets, A, B and C with bound ligands and conservation of the C pocket.

(A) Structure I: Sir2Af2 bound to nicotinamide and non-productive NAD⁺.

(B) Structure II: Sir2Af2 bound to nicotinamide and ADP-ribose.

(C) Structure III: Sir2Tm bound to nicotinamide and acetylated p53 peptide.

(D) Multiple sequence alignment of eight sirtuins showing highly conserved residues and conserved residues. Residues that contact nicotinamide or contact NAD⁺ bound in the productive conformation are indicated above the alignment. The Asp that confers NAAD-dependent deacetylation activity and nicotinic acid sensitivity when mutated to Asn is marked.

FIG. 4. Stereo figures of the interactions of nicotinamide and NAD⁺ bound in the C pocket of sirtuins. The nicotinamide rotamer shown in panels A-C was chosen to maximize favorable interactions, as described in the text.

(A) Structure I: C pocket of Sir2Af2 bound to nicotinamide and non-productive NAD⁺.

(B) Structure II: C pocket of Sir2Af2 bound to nicotinamide and ADP ribose.

(C) Structure III: C pocket of Sir2Tm bound to nicotinamide and acetyl-lysine from the p53-bound peptide.

(D) C pocket of Sir2Af2 (white) bound to productive NAD⁺.

FIG. 5. Effects of the D101N mutation on the enzymatic activity and regulation of Sir2Tm.

(A) Schematic representation of the H-bond network observed between productive NAD⁺ and Asp101 and the hypothetical H-bond network between productive NAAD and the substituted Asn101.

(B) Initial rates of NAD⁺-dependent and NAAD-dependent deacetylation activities of the wild type (circles) and D101N mutant (squares) Sir2Tm. (C) Inhibition of Sir2Tm wild type's NAD⁺-dependent deacetylation activity (circles) and the Sir2Tm-D101N mutant's NAD⁺-dependent (squares) and NAAD-dependent (triangles) deacetylation activities by nicotinamide and nicotinic acid.

(D) TLC plate showing the products of the base exchange reactions of Sir2Tm wild type and D101N mutant with unlabeled NAD⁺, acetylated p53 peptide and either ¹⁴C-nicotinamide or ¹⁴C-nicotinic acid. The migration of NAD⁺ and NAAD are marked with arrows.

FIG. 6. Structure-based mechanism of the enzymatic activity and regulation of sirtuins.

(i) NAD⁺ binds in a productive conformation in the C pocket, making hydrogen bonds with the rigid wall of the pocket, which promotes NAD⁺ cleavage.

(ii) The produced O-alkyl amidate intermediate in the extended conformation can reform NAD⁺ with a reactive nicotinamide in the C pocket unless the nicotinamide is entrapped by flipping, or the intermediate shifts to a contracted conformation.

(iii) The entrapped nicotinamide buries its N1 against residues in the flexible wall of the C pocket, thereby preventing it from reacting with the O-alky amidate, even if this intermediate is in the extended conformation. From this position nicotinamide can either flip out of entrapment, or be released by the enzyme.

(iv) The empty C pocket will have certain affinity for nicotinamide in the cell.

(v) By shifting to a contracted conformation, the O-alkyl amidate intermediate is shielded by Phe33 and brings its 2′ and 3′ OH groups closer to His116, both of which promote deacetylation.

(vi) The empty C pocket will have certain affinity for nicotinamide.

FIG. 7. Proposed alternative conformations of the O-alkyl amidate intermediate.

(A) The contracted conformation of the O-alkyl amidate intermediate is too far from the nicotinamide in the C pocket and is shielded by Phe33. However, in this conformation, the 2′ and 3′OH groups of the intermediate are at a suitable distance and orientation from His116 to promote deacetylation. This conformation was modeled from the structure of Hst2 bound to acetylated histone peptide and 2′O-acetyl-ADP ribose.

(B) The extended conformation of the O-alkyl amidate intermediate is further from His116, closer to the reactive nicotinamide in the C pocket and exposed by Phe33, thereby promoting nicotinamide exchange. This intermediate and the reactive nicotinamide were modeled from the position of NAD⁺ in structure V and the acetyl-lysine position in the structures of Sir2Af2 and Sir2Tm bound to acetylated peptide.

FIG. 8. Crystal structure data of archaeal and bacterial sirtuins bound to nicotinamide.

FIG. 9. Ribbon diagram of the asymmetric unit of the Sir2Af2 crystal, containing five sirtuin monomers, four NAD+, one ADP-ribose, nine PEG and five nicotinamide molecules, three bound in the C pocket and two non-specifically bound.

FIG. 10. Average and overall B factors of ligands and structures.

### DEFINITIONS

In order that the invention may be more readily understood, certain terms are first defined and collected here for convenience.

The term “Sir2” refers to the silent information regulator family of proteins, also known as sirtuins. This family includes both mammalian and non-mammalian proteins. “Sir2” also means silent information regulator 2, or any of its orthologs or paralogs, now known or later discovered, as would be understood by the skilled artisan.

“Sir2 activity” refers to one or more activity of Sir2, e.g., deacetylation of p53 or histone proteins. “Modulating Sir2 activity” refers to increasing or decreasing one or more activity of Sir2, e.g., deacetylation of p53 or histone proteins, e.g., by altering the binding affinity of Sir2 and p52, introducing exogenous Sir2 (e.g., by expressing or adding purified recombinant Sir2), increasing or decreasing levels of NAD and/or an NAD analog (e.g., 3-aminobenzamide, 1,3-dihydroxyisoquinoline), and/or increasing or decreasing levels of a Sir2 inhibitor, e.g., nicotinamide and/or a nicotinamide analog.

As used herein, “NAD⁺” means nicotinamide adenine dinucleotide.

The term “NAD+ dependent deacetylase” refers to a protein that removes the acetyl groups from a lysine residue of another protein, wherein the deacetylation is coupled to NAD (nicotinamide adenosine dinucleotide) cleavage.

The term “administration” or “administering” includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively.

The term “antineoplastic agent” refers to a means for inhibiting or combating the undesirable growth of biological tissue. Antineoplastic agents include, but are not limited to, antiangiogenic and antivascular agents, antimetabolites, antifolates and other inhibitors of DNA synthesis, antisense oligonucleotides, biological response modifiers, DNA-alkylating agents, DNA intercalators, DNA repair agents, growth factor receptor kinase inhibitors, hormone agents, immunoconjugates, microtubule disruptors and topoisomerase I/II inhibitors. Antineoplastic agents can also include cyclophosphamide, triethylenephosphoramide, triethylenethio phosphoramide, flutamide, altretamine, triethylenemelamine, trimethylolmelamine, meturedepa, uredepa, aminoglutethimide, L-asparaginase, BCNU, benzodepa, bleomycin, busulfan, camptothecin, capecitabine, carboquone, chlorambucil, cytarabine, dactinomycin, daunomycin, daunorubicin, docetaxol, doxorubicin, epirubicin, estramustine, dacarbazine, etoposide, fluorouracil, gemcitabine, hydroxyurea, ifosfamide, improsulfan, mercaptopurine, methotrexate, mitomycin, mitotane, mitoxantrone, novembrichin, paclitaxel, piposulfan, plicamycin, prednimustine, procarbazine, tamoxifen, temozolomide, teniposide, thioguanine, thiotepa, UFT, uracil mustard, vinblastine, vincristine, vinorelbine and vindesine.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “autoimmune disease” or “autoimmune disorder” refers to the condition where the immune system attacks the host's own tissue(s). In an autoimmune disease, the immune tolerance system of the patient fails to recognize self antigens and, as a consequence of this loss of tolerance, brings the force of the immune system to bear on tissues which express the antigen. Autoimmune disorders include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The language “biological activities” includes all genomic and non-genomic activities elicited by these compounds.

The term “cancer” refers to a malignant tumor of potentially unlimited growth that expands locally by invasion and systemically by metastasis. The term “cancer” also refers to the uncontrolled growth of abnormal cells. Specific cancers are selected from, but not limited to, rhabdomyosarcomas, chorio carcinomas, glioblastoma multiformas (brain tumors), bowel and gastric carcinomas, leukemias, ovarian cancers, prostate cancers, lymphomas, osteosarcomas or cancers which have metastasized.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

The phrase “deacetylating p53” refers to the removal of one or more acetyl groups from p53 that is acetylated on at least one amino acid residue.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

A therapeutically effective amount of compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “flexible loop,” refers to a highly conserved 15-30 amino acid group in the front wall of the C pocket in residues in sirtuins, which adopts a variety of conformations in different crystal structures and is in some cases partially disordered.

The term “gene” refers to any segment of DNA associated with a biological function. Thus, genes include coding sequences and/or the regulatory sequences required for their expression. Genes also include nonexpressed DNA segments that, for example, form recognition sequences for other proteins.

The term “genetic blood disease” refers to a hereditary disease of the blood that includes, but is not limited to, hyperproliferative diseases, thalassaemias and sickle cell disease.

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The terms “hyperproliferative” and “neoplastic” are used interchangeably, and include those cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Other “disorders” include an immune disorder, e.g., an autoimmune disorder, such as type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease; obesity, or transplant rejection, such as graft versus host disease (GVHD).

The term “immune response” includes T and/or B cell responses, e.g., cellular and/or humoral immune responses. The claimed methods can be used to reduce both primary and secondary immune responses. The immune response of a subject can be determined by, for example, assaying antibody production, immune cell proliferation, the release of cytokines, the expression of cell surface markers, cytotoxicity, and the like.

The language “improved biological properties” refers to any activity inherent in a compound of the invention that enhances its effectiveness in vivo. In a preferred embodiment, this term refers to any qualitative or quantitative improved therapeutic property of a compound, such as reduced toxicity.

The terms “inhibition”, “inhibits” and “inhibitor” refer to a method of prohibiting a specific action or function.

The language “inhibiting the growth” of the neoplasm includes the slowing, interrupting, arresting or stopping its growth and metastases and does not necessarily indicate a total elimination of the neoplastic growth.

The term “isomers” or “stereoisomers” refers to compounds which have identical chemical constitution, but differ with regard to the arrangement of the atoms or groups in space.

The term “ligand binding domain” or “compound binding domain” refers to a region of a protein, enzyme, or gene that binds to a ligand or a compound, selective for that particular site.

The term “leukemia” is intended to have its clinical meaning, namely, a neoplastic disease in which white corpuscle maturation is arrested at a primitive stage of cell development. The condition may be either acute or chronic. Leukemias are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

The term “leukemic cancer” refers to all cancers or neoplasias of the hemopoietic and immune systems (blood and lymphatic system). Chronic myelogenous leukemia (CML), also known as chronic granulocytic leukemia (CGL), is a neoplastic disorder of the hematopoietic stem cell. The term “leukemia” is art recognized and refers to a progressive, malignant disease of the blood-forming organs, marked by distorted proliferation and development of leukocytes and their precursors in the blood and bone marrow.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

“Modulating p53 activity” refers to increasing or decreasing p53 activity, e.g., p-53 mediated apoptosis, cell cycle arrest, and/or senescence, e.g. by altering the acetylation or phosphorylation status of p53.

The term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangably, as their context will reveal, referring to generally to cells experiencing abnormal cell growth rates. Neoplasias and hyperplasias include “tumors,” which may be either benign, premalignant or malignant.

A “nicotinamide analog” as used herein refers to a compound (e.g., a synthetic or naturally occurring chemical, drug, protein, peptide, small organic molecule) which possesses structural similarity to component groups of nicotinamide or functional similarity (e.g., reduces Sir2 deacetylation activity of p53).

The term “non-direct interaction” refers to any interactions that are not ionic nor covalent, such as hydrogen bonding.

The language “non-genomic” activities include cellular (e.g., calcium transport across a tissue) and subcellular activities (e.g., membrane calcium transport opening of voltage-gated calcium channels, changes in intracellular second messengers) elicited by compounds in a responsive cell. Electrophysiological and biochemical techniques for detecting these activities are known in the art.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

A “peptide” is a sequence of at least two amino acids. Peptides can consist of short as well as long amino acid sequences, including proteins.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g. benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The term “protein” refers to series of amino acid residues connected one to the other by peptide bonds between the alpha-amino and carboxy groups of adjacent residues. In general, the term “protein” is used to designate a series of greater than 50 amino acid residues connected one to the other.

The language “reduced toxicity” is intended to include a reduction in any undesired side effect elicited by a compound when administered in vivo.

The term “sarcoma” is art recognized and refers to malignant tumors of mesenchymal derivation.

The terms “silence”, “silencing” and “silenced” refers to a mechanism by which gene expression in particular regions of the genome are repressed.

The term “subject” refers to animals such as mammals, including, but not limited to, primates (e.g., humans), cows, sheep, goats, horses, dogs, cats, rabbits, rats, mice and the like. In certain embodiments, the subject is a human.

The term “sulfhydryl” or “thiol” means —SH.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

The term “therapeutically effective amount” refers to that amount of the compound being administered sufficient to prevent development of or alleviate to some extent one or more of the symptoms of the condition or disorder being treated.

The terms “treating” and “treatment” refer to a method of alleviating or abating a disease and/or its attendant symptoms.

The term “tumor suppressor gene” refers to a gene that acts to suppress the uncontrolled growth of a cancer, such as a tumor.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

### Methods of the Invention

In one aspect, the invention provides a method for identifying a compound which modulates the activity of a Sir2 enzyme, the method comprising: a) contacting a Sir2 enzyme with a compound under conditions suitable for modulation of the activity of the Sir2 enzyme; and b) detecting modulation of the activity of the Sir2 enzyme by the compound; wherein the compound is capable of interacting with the C-pocket of the Sir2 enzymes.

In another aspect, the invention provides a method for identifying a compound which modulates the binding of a Sir2 enzyme, the method comprising: a) contacting a Sir2 enzyme with a compound under conditions suitable for modulation of the binding of the Sir2 enzyme; and b) detecting modulation of the binding of the Sir2 enzyme by the compound; wherein the compound is capable of interacting with the C-pocket of the Sir2 enzymes.

In still another aspect, the invention provides a method of modulating the Sir2 activity in a subject, the method comprising administering to the subject a compound identified by: a) contacting a Sir2 enzyme with a compound under conditions suitable for modulation of the activity of the Sir2 enzyme; and b) detecting modulation of the activity of the Sir2 enzyme by the compound; wherein the compound is capable of interacting with the C-pocket of the Sir2 enzymes.

In yet another aspect, the invention provides a method for identifying a compound which modulates the activity of a Sir2 enzyme, the method comprising: a) contacting a Sir2 enzyme with a compound under conditions suitable for modulation of the activity of the Sir2 enzyme; and b) detecting modulation of the activity of the Sir2 enzyme by the compound; wherein the compound is capable of interacting with the flexible loop of the Sir2 enzyme.

In another aspect, the invention provides a method for identifying a compound which modulates the binding of Sir2 enzymes, the method comprising: a) contacting a Sir2 enzyme with a compound under conditions suitable for modulation of the binding of the Sir2 enzyme; and b) detecting modulation of the binding of the Sir2 enzyme by the compound; wherein the compound is capable of interacting with the flexible loop of the Sir2 enzyme.

In still another aspect, the invention provides a method of modulating the Sir2 activity in a subject, the method comprising administering to the subject a compound identified by: a) contacting a Sir2 enzyme with a compound under conditions suitable for binding or modulation of the activity of the Sir2 enzyme; and b) detecting modulation of the activity or the binding of the Sir2 enzyme by the compound; wherein the compound is capable of interacting with the flexible loop of the Sir2 enzyme.

In other aspects, the invention further comprises the intention of identifying compounds that bind to the flexible loop. In certain embodiments, the invention further comprises intention of identifying compounds that bind to a highly conserved region of the flexible loop. In certain embodiments, the conserved region is described in FIG. 3D.

In certain embodiments, the interaction of the of the compound with the Sir2 enzyme is a binding interaction. In one embodiment, the binding interaction is ionic, covalent, or a non-direct interaction. Preferably, the interaction is covalent.

In other embodiments, the interaction of the compound with the flexible loop causes a conformational change of the Sir2 enzyme. In one embodiment, the flexible loop becomes rigid.

In one embodiment, the compound has a binding interaction with a conserved aspartic acid in the C pocket of the Sir2 enzyme (Asp 103). In another embodiment, the compound has a binding interaction with a conserved isoleucine in the C pocket of the Sir2 enzyme (Ile102). In still another embodiment, the compound has a binding interaction with a highly conserved residues in the C-pocket, selected from Ala 24, Ile 32, Phe 35 or Ile 102.

In certain embodiments, the invention contemplates synthesizing compounds identified in steps a) and b) from above.

In another embodiments, the modulation of the activity of the Sir2 enzyme is detected by direct binding of the compound to the Sir2 enzyme. In certain embodiments, the modulation of the activity of the Sir2 enzyme is inhibition of the activity of the Sir2 enzyme. In other embodiments, the modulation of the activity of the Sir2 enzyme is stimulation of the activity of the Sir2 enzyme.

In another embodiment, the modulation of the activity of the Sir2 enzyme is detected by use of an assay for deacetylation activity. In a further embodiment, the detection of the Sir2 enzyme activity modulation is carried out by attaching a detection complex to the Sir2 enzyme.

In another embodiment, the detection complex comprises a fluorophore and a fluorescence quencher. In a further embodiment, the interaction between the compound and the Sir2 enzyme is determined by cleaving the enzyme from the compound and observing a fluorescence. Preferably, the interaction is determined by a Fluor de lys-SirT1 assay.

In another aspect, the invention provides a method for identifying a compound which modulates the activity of Sir2 enzymes, the method comprising: a) contacting a Sir2 enzyme, attached to a fluorophore and a fluorescence quencher, with a test compound at 37° C.; and

b) detecting the fluorescence of the Sir2 enzyme by the test compound at 340 nm; wherein the test compound binds to the flexible loop region of the Sir2 enzymes.

In another aspect, the invention provides a method for identifying a compound which modulates the binding or activity of Sir2 enzymes, the method comprising: a) creating a computer model of the structure of the flexible loop of a Sir2 enzyme based on the crystal structure of the Sir2 enzyme; b) introducing a compound to the flexible loop region of the Sir2 enzyme; and c) determining from computer calculations whether the compound interacts with the flexible loop region of the Sir2 enzyme.

In one embodiment, the invention further comprises: a) contacting a Sir2 enzyme with a compound determined in claim 26 under conditions suitable for modulation of the activity of the Sir2 enzyme; and b) detecting modulation of the activity of the Sir2 enzyme by the compound.

In another embodiment, detecting the modulation of the activity is carried out using a Fluor de lys-SirT1 assay. In still another embodiment, the invention provides for synthesizing compounds identified in steps a) and b).

In another aspect, the invention provides a method for identifying a compound which modulates the binding or activity of Sir2 enzymes, the method comprising: a) creating a computer model of the structure of the flexible loop of a Sir2 enzyme based on the three-dimensional structure coordinates of any of FIG. 8; Table 1, of the Sir2 enzyme; b) introducing a compound to the flexible loop region of the Sir2 enzyme; and c) determining from computer calculations whether the compound interacts with the flexible loop region of the Sir2 enzyme.

In a further embodiment, the invention provides for synthesizing the compound. In another further embodiment, the invention provides for testing the compound for biological activity.

In another embodiment, the compound is designed de novo.

In one embodiment, the compound is designed from a known ligand of Sir2. In a further embodiment, the compound is designed from a nicotinamide derivative. In a further embodiment, the compound is designed from a derivative of NAD+.

In one embodiment, the compound identified comprises polyphenol compounds or an analog or derivative thereof selected from the group consisting of stilbenes, chalcones, and flavones, or a non-polyphenol dipyridamole compound. In a further embodiment, the polyphenol compound or non-polyphenol dipyridamole compound is selected from the group consisting of 3,5-dihydroxy-4′-chloro-trans-stilbene, dipyridamole, 3,5-dihydroxy-4′ethyl-trans-stilbene, 3,5-dihydroxy-4′-isopropyl-trans-stilbene, 3,5-dihydroxy-4′-methyl-trans-stilbene, resveratrol, 3,5-dihydroxy-4′thiomethyl-trans-stilbene, 3,5-dihydroxy-4′-carbomethoxy-1-trans-stilbene, isoliquiritgenin, 3,5-dihydro-4′nitro-trans-stilbene, 3,5-dihydroxy-4′azido-trans-stilbene, piceatannol, 3-methoxy-5-hydroxy-4′acetamido-trans-stilbene, 3,5-dihydroxy-4′acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′carboxy-trans-1-stilbene, and 3,4′-dihydroxy-5-acetoxy-trans-stilbene, or an analog or derivative thereof.

In one embodiment, the compound further comprises a transcription factor.

In another aspect, the invention provides a method of treating a disorder in a subject, comprising administering to said subject in need thereof, an effective amount of a compound identified, such that said subject is treated for said disorder.

In certain embodiments, the disorder is age related disorders, cancer or genetic blood diseases, silenced tumor suppressor genes, B-cell-derived non-Hodgkin lymphomas, diffuse large B-cell lymphomas, thalassaemias, sickle cell disease, autoimmune diseases, inflammatory diseases, viral infections, diseases that are associated with a decrease in cell death due to hyperactive apoptosis, cell growth, aging, cell apoptosis, DNA-damaging ionizing radiation, ionizing radiation, metabolic diseases, hyperlipidemia, hypercholesterolemia or type 2 diabetes.

In a further embodiment, the age-related disorder is slow replicative aging, cataracts, hypermelanosis, osteoporosis, cerebral cortical atrophy, lymphoid depletion, thymic atrophy, diabetes type II, atherosclerosis, heart disease, lordokyphosis, absence of vigor, lymphoid atrophy, dermal thickening and subcutaneous adipose tissue, atrophy of intestinal villi, skin ulceration, amyloid deposits, and joint diseases. In another embodiment, the diseases that are associated with a decrease in cell death due to hyperactive apoptosis consist of the following: AIDS, neurodegenerative disease, hematologic diseases, and tissue damage.

In certain embodiments, the contacting takes place in a cell. In other embodiments, the cell is in a mammal. In another embodiment, the cell is from a mammal. In further embodiments, the mammal is a human or a rodent. In still other embodiments, the contacting takes place in a cell-free system. In other embodiments, the cell is in vitro.

Sir2 enzymes deacetylate peptides other than histones. Examples of such peptides are acetylated p53 and fragments thereof. The skilled artisan would expect that Sir2 enzymes deacetylate any acetylated peptide of at least two amino acids, wherein at least one of the amino acids comprises a lysine residue that is acetylated at the ε-amino moiety. This finding would lead the skilled artisan to believe that Sir2 enzymes have a much broader role in regulating transcription than was previously appreciated, since it is now understood that Sir2 enzymes can deacetylate any acetylated protein.

It is believed that any acetylated peptide of at least two amino acids can usefully serve as a substrate for Sir2, provided at least one of the amino acids is a lysine residue that is acetylated at the ε-amino moiety. The acetylated peptide can comprise any number of amino acids, including three, five, ten, fifteen, eighteen, or more amino acid residues. As is known, there is no particular sequence that is preferred, although most deacetylation occurs in a pair of basic amino acids.

The described enzyme reaction is expected to be similar or the same for many Sir2 enzymes, including but not limited to Sir2Af2, human Sir2A, yeast Sir2p, Sir2Tm from Thermotoga maritima, and cobB, from Salmonella typhimurium. Useful enzymes include those derived from prokaryotes, including archaeal bacteria and eubacteria, and those derived from prokaryotes, including yeast and humans.

In related embodiments, the invention is directed to methods of stimulating or inhibiting Sir2 enzymes. The methods comprise combining the Sir2 enzyme with compound found by the method described above. The Sir2 enzyme to be inhibited can be within a living cell, wherein the inhibitor is inserted into the cell by any of a number of methods, depending on the chemical characteristics of the inhibitor, as is known in the art.

Additional embodiments of the invention are directed to methods of deacetylating an acetylated peptide. The methods comprise combining the peptide with a Sir2 enzyme. In certain aspects of these embodiments, the acetylated peptide is not a histone. As with previously described methods, any Sir2 enzyme that produces 2′/3′-O-acetyl-ADP-ribose is useful for these methods; the methods would also be expected to be useful for the deacetylation of any acetylated peptide that consists of at least two amino acids, wherein at least one of the amino acids comprises a lysine residue that is acetylated at the ε-amino moiety.

The invention is also directed to methods of stimulating or inhibiting the deacetylation of an acetylated peptide. These methods are useful for stimulating or inhibiting deacetylation of any acetylated peptide in vitro or in vivo. Although these methods are useful in vitro, in preferred embodiments the acetylated peptide is in a living cell. The living cell can be a prokaryotic cell or, preferably, a eukaryotic cell. The eukaryotic cell can be a mammalian cell, optionally in a living mammal, such as a human. Using a radiolabeled Sir2 substrate can simplify these methods.

Accordingly, the present invention provides methods of identifying agents that can be used for reducing the life span of cells, such as to treat conditions that may benefit from reducing the life span of certain cells.

In a preferred embodiment the method further comprises preparing a supplemental crystal containing at least a portion of a Sir2 family member comprising the C pocket bound to the potential agent. Preferably the supplemental crystal effectively diffracts X-rays for the determination of the atomic coordinates to a resolution of better than 5.0 Angstroms, more preferably to a resolution equal to or better than 3.5 Angstroms, and even more preferably to a resolution equal to or better than 3.3 Angstroms. The three-dimensional coordinates of the supplemental crystal are then determined with molecular replacement analysis and a second generation agent is selected by performing rational drug design with the three-dimensional coordinates determined for the supplemental crystal. Preferably the selection is performed in conjunction with computer modeling. The second generation agent can be an analog of nicotinamide.

As should be readily apparent the three-dimensional structure of a supplemental crystal can be determined by molecular replacement analysis or multiwavelength anomalous dispersion or multiple isomorphous replacement. A compound can then be selected based on the three-dimensional structure determined for the supplemental crystal, preferably in conjunction with computer modeling. The candidate drug can then be tested in a large number of drug screening assays using standard biochemical methodology exemplified herein.

The method can further comprise contacting the second generation agent with a Sir2 family member or portion thereof of a different species and determining the activity of the Sir2 family member or portion thereof of the other species. A potential agent is then identified as an agent for use as an essentially specific agonist or inhibitor of a Sir2 family member of a first species when there is significantly less change (a factor of two or more) in the activity of the Sir2 family member of other species relative to that observed for the Sir2 family member of the first species.

In another aspect, the present invention provides a method for making an agonist or inhibitor of a Sir2 family member, the method including chemically or enzymatically synthesizing a chemical entity to yield an agonist or inhibitor of the activity of a Sir2 family member, the chemical entity having been designed during a computer-assisted process.

### Compounds Identified by the Methods of the Invention

The invention includes sequences and variants that include one or more substitutions, e.g., between one and six substitutions, e.g., with respect to a naturally-occurring protein. Whether or not a particular substitution will be tolerated can be determined by a method described herein. One or more or all substitutions may be conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison methodology such as BLAST or BLAST 2.0. Such sequences are then said to be “substantially identical.” This definition also refers to, or may be applied to, the complement of a test nucleic acid sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is at least 50 or 100 amino acids or nucleotides in length.

In some embodiments, the method is repeated one or more times such that, e.g., a library of test compounds can be evaluated. In an related embodiment, the evaluating of the interaction with the test compound and the Sir2 or the transcription factor, e.g., p53, is repeated, and the evaluating of the rate of aging is selectively used for compounds for which an interaction is detected. Possible test compounds include, e.g., small organic compounds, peptides, antibodies, and nucleic acid molecules.

Small molecule compounds may also be developed by generating a library of molecules, selecting for those molecules which act as ligands for a specified target, (using protein functional assays, for example), and identifying the selected ligands. See, e.g., Kohl et al., Science 260: 1934, 1993. Techniques for constructing and screening combinatorial libraries of small molecules or oligomeric biomolecules to identify those that specifically bind to a given receptor protein are known. Suitable oligomers include peptides, oligonucleotides, carbohydrates, nonoligonucleotides (e.g., phosphorothioate oligonucleotides; see Chem. and Engineering News, page 20, Feb. 7, 1994) and nonpeptide polymers (see, e.g., “peptoids” of Simon et al., Proc. Natl. Acad. Sci. USA 89 9367, 1992). See also U.S. Pat. No. 5,270,170 to Schatz; Scott and Smith, Science 249: 386-390, 1990; Devlin et al., Science 249: 404-406, 1990; Edgington, BI0/Technology, 11: 285, 1993. Libraries may be synthesized in solution on solid supports, or expressed on the surface of bacteriophage viruses (phage display libraries).

Examples of methods for the synthesis of molecular libraries can be found in the art, for example in: DeWitt et al., Proc. Natl. Acad. Sci. U.S.A. 90: 6909, 1993; Erb. et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and in Gallop et al., J. Med. Chem. 37:1233, 1994.

Libraries of compounds may be presented in solution (e.g., Houghten, Biotechniques 13: 412-421, 1992), or on beads (Lam, Nature 354: 82-84, 1991), chips (Fodor, Nature 364: 555-556, 1993), bacteria (Ladner U.S. Pat. No. 5,223,409), spores (Ladner U.S. Pat. No. '409), plasmids (Cull et al., Proc Natl Acad Sci USA 89: 1865-1869, 1992) or on phage (Scott and Smith, Science 249: 386-390, 1990); (Devlin, Science 249: 404-406, 1990); (Cwirla et al., Proc. Natl. Acad. Sci. U.S.A. 87: 6378-6382, 1990); (Felici, J. Mol. Biol. 222: 301-310, 1991); (Ladner supra.).

The compounds tested as modulators of Sir2 or p53 can be any small chemical compound, or a biological entity, such as a protein, e.g., an antibody, a sugar, a nucleic acid, e.g., an antisense oligonucleotide or a ribozyme, or a lipid. Alternatively, modulators can be genetically altered versions of Sir2 or p53. Typically, test compounds will be small chemical molecules and peptides, or antibodies, antisense molecules, or ribozymes. Essentially any chemical compound can be used as a potential modulator or ligand in the assays of the invention, although most often compounds that can be dissolved in aqueous or organic solutions are used. In one preferred embodiment, high throughput screening methods known to one of ordinary skill in the art involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. Moreover, a combinatorial library can be designed to sample a family of compounds based on a parental compound, e.g., based on the chemical structure of NAD or nicotinamide.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

In one embodiment, the invention provides solid phase based in vitro assays in a high throughput format, e.g., where each assay includes a cell or tissue expressing Sir2 and/or p53. In a high throughput assays, it is possible to screen up to several thousand different modulators or ligands in a single day.

Candidate Sir2- or p53-interacting molecules encompass many chemical classes. They can be organic molecules, preferably small organic compounds having molecular weights of 50 to 2,500 Daltons. The candidate molecules comprise functional groups necessary for structural interaction with proteins, particularly hydrogen bonding, for example, carbonyl, hydroxyl, and carboxyl groups. The candidate molecules can comprise cyclic carbon or heterocyclic structures and aromatic or polyaromatic structures substituted with the above groups. In one embodiment, the candidate molecules are structurally and/or chemically related to NAD or to nicotinamide.

Nucleic acid molecules may also act as ligands for receptor proteins. See, e.g., Edgington, BIO/Technology 11: 285, 1993. U.S. Pat. No. 5,270,163 to Gold and Tuerk describes a method for identifying nucleic acid ligands for a given target molecule by selecting from a library of RNA molecules with randomized sequences those molecules that bind specifically to the target molecule. A method for the in vitro selection of RNA molecules immunologically cross-reactive with a specific peptide is disclosed in Tsai et al., Proc. Natl. Acad. Sci. USA 89: 8864, (1992); and Tsai et al. Immunology 150:1137, (1993). In the method, an antiserum raised against a peptide is used to select RNA molecules from a library of RNA molecules; selected RNA molecules and the peptide compete for antibody binding, indicating that the RNA epitope functions as a specific inhibitor of the antibody-antigen interaction.

Antibodies that are both specific for a target gene protein and that interfere with its activity may be used to inhibit target gene function. Such antibodies may be generated using standard techniques, against the proteins themselves or against peptides corresponding to portions of the proteins. Such antibodies include but are not limited to polyclonal, monoclonal, Fab fragments, single chain antibodies, chimeric antibodies, and the like. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Sambrook et al., Eds., Molecular Cloning: A Laboratory Manual, 2nd ed., Cold Spring Harbor Laboratory Press, (1989), or Ausubel, F. M. et al., eds. Current Protocols in Molecular Biology (1994).

Alternatively, single chain neutralizing antibodies that bind to intracellular target gene epitopes may also be administered. Such single chain antibodies may be administered, for example, by expressing nucleotide sequences encoding single-chain antibodies within the target cell population by utilizing, for example, techniques such as those described in Marasco et al., Proc. Natl. Acad. Sci. USA 90: 7889-7893 (1993).

Oligonucleotides may be designed to reduce or inhibit mutant target gene activity. Techniques for the production and use of such molecules are well known to those of ordinary skill in the art. Antisense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Antisense oligonucleotides are preferably 10 to 50 nucleotides in length, and more preferably 15 to 30 nucleotides in length. An antisense compound is an antisense molecule corresponding to the entire Sir2 or p53 mRNA or a fragment thereof.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the well known catalytic sequence responsible for mRNA cleavage disclosed, for example, in U.S. Pat. No. 5,093,246. Within the scope of this disclosure are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites that include the sequences GUA, GUU, and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules used in triple helix formation for the inhibition of transcription should be single stranded and composed of deoxyribonucleotides. The base composition of these oligonucleotides are designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences targeted for triple helix formation may be increased by creating a “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The antisense, ribozyme, and/or triple helix molecules described herein may reduce or inhibit the transcription (triple helix) and/or translation (antisense, ribozyme) of mRNA produced by both normal and mutant target gene alleles. If it is desired to retain substantially normal levels of target gene activity, nucleic acid molecules that encode and express target gene polypeptides exhibiting normal activity may be introduced into cells via gene therapy methods that do not contain sequences susceptible to whatever antisense, ribozyme, or triple helix treatments are being utilized. Alternatively, it may be preferable to coadminister normal target gene protein into the cell or tissue in order to maintain the requisite level of cellular or tissue target gene activity.

Antisense RNA and DNA, ribozyme, and triple helix molecules may be prepared by any method known in the art for the synthesis of DNA and RNA molecules. These include techniques for chemically synthesizing oligodeoxyribonucleotides and oligoribonucleotides, for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules may be introduced as a means of increasing intracellular stability and half-life. Possible modifications include but are not limited to the addition of flanking sequences of ribonucleotides or deoxyribonucleotides of the 5′ and/or 3′ ends of the molecule or the use of phosphorothioate or 2′ O-methyl rather than phosphodiesterase linkages within the oligodeoxyribonucleotide backbone.

Delivery of antisense, triplex agents, ribozymes, and the like can be achieved using a recombinant expression vector such as a chimeric virus or a colloidal dispersion system or by injection. Useful virus vectors include adenovirus, herpes virus, vaccinia, and/or RNA virus such as a retrovirus. The retrovirus can be a derivative of a murine or avian retrovirus such as Moloney murine leukemia virus or Rous sarcoma virus. All of these vectors can transfer or incorporate a gene for a selectable marker so that transduced cells can be identified and generated. The specific nucleotide sequences that can be inserted into the retroviral genome to allow target specific delivery of the retroviral vector containing an antisense oligonucleotide can be determined by one of skill in the art.

Another delivery system for polynucleotides is a colloidal dispersion system. Colloidal dispersion systems include macromolecular complexes, nanocapsules, microspheres, beads, and lipid-based systems including oil-in-water emulsions, micelles, mixed micelles and liposomes. A preferred colloidal delivery system is a liposome, an artificial membrane vesicle useful as in vivo or in vitro delivery vehicles. The composition of a liposome is usually a combination of phospholipids, usually in combination with steroids, particularly cholesterol.

In some embodiments, the interaction between the test compound and the Sir2 or transcription factor, e.g., p53, is evaluated in vitro, e.g., using an isolated polypeptide. The Sir2 or transcription factor, e.g., p53, polypeptide can be in solution (e.g., in a micelle) or bound to a solid support, e.g., a column, agarose beads, a plastic well or dish, or a chip (e.g., a microarray). Similarly, the test compound can be in solution or bound to a solid support.

In another aspect, the invention features a method of evaluating a protein. In some embodiments, the candidate protein is identified by amplification of the gene or a portion thereof encoding the candidate protein, e.g., using a method described herein, e.g., PCR amplification or the screening of a nucleic acid library. In preferred embodiments, the candidate protein is identified by searching a database, e.g., searching a sequence database for protein sequences homologous to Sir2.

In preferred embodiments, the candidate protein is a human protein. In other embodiments, the candidate protein is a mammalian protein, e.g., a mouse protein. In other embodiments, the protein is a vertebrate protein, e.g., a fish, bird or reptile protein, or an invertebrate protein, e.g., a worm or insect protein. In still other embodiments, the protein is a eukaryotic protein, e.g., yeast protein. The assay can be conducted either in the solid phase or in the liquid phase.

### Treatment of Diseases

The present invention provides in one aspect a method for identifying compounds useful for the treatment of cancer or genetic blood diseases, comprising the step of determining whether the compound inhibits the deacetylase activity of a NAD+ dependent deacetylase. In a related aspect of the present invention, the method for treating cancer or genetic blood diseases comprises the step of administering to a subject in need thereof, a therapeutically effective amount of a compound that inhibits the deacetylase activity of a NAD+ dependent deacetylase.

In a preferred aspect of the present invention, the identified compounds are useful for the treatment of silenced tumor suppressor genes, B-cell-derived non-Hodgkin lymphomas and diffuse large B-cell lymphomas. In another preferred aspect of the present invention, the identified compounds are useful for the treatment of thalassaemias and sickle cell disease.

In another preferred aspect of the present invention, the NAD+ dependent deacetylase is a member of the Sir2 family of proteins. In a more preferred aspect, the member of the Sir2 family of proteins is selected from the group consisting of Sir2p and Sir2α. In a most preferred aspect, the member of the Sir2 family of proteins is Sir2α.

In another aspect of the present invention, a method is provided for identifying compounds which will be useful for the treatment of cancer or genetic blood diseases, comprising the step of determining whether the compound inhibits the NAD+ dependent deacetylase activity of a member of the Sir2 family of proteins. In a preferred aspect of the present invention, the method for treating cancer or genetic blood diseases comprises the step of administering to a subject in thereof, a therapeutically effective amount of a compound that inhibits the NAD+ dependent deacetylase activity of a member of the Sir2 family of proteins.

In another preferred aspect of the present invention, a method is provided for activating a silenced gene in a cell, comprising contacting the cell with an effective amount of a compound which is capable of inhibiting the NAD+ dependent deacetylase activity of a member of the Sir2 family of proteins.

In still another preferred aspect of the present invention, a method is provided for promoting p53-dependent apoptosis of a cell comprising contacting the cell with an effective amount of a compound which is capable of inhibiting the NAD+ dependent deacetylase activity of a member of the Sir2 family of proteins.

In a further aspect of the present invention, a method is provided for inhibiting BCL6 transcriptional repressor activity, comprising contacting a cell with an effective amount of a compound which is capable of inhibiting the NAD+ dependent deacetylase activity of a member of the Sir2 family of proteins.

Several of the proteins in this class play an important role in the silencing of genes. In one aspect, the deacetylation of histone by a protein in the Sir2 class, can lead to the silencing of tumor suppressor genes. In another aspect, the deacetylation of the p53 tumor suppressor gene by a protein in the Sir2 class, reduces p53-dependent apoptosis. Diseases in which apoptosis is involved include diseases that are associated with an increase in cell survival due to inhibition of apoptosis, such as cancer, autoimmune diseases, inflammatory diseases and viral infections and diseases that are associated with a decrease in cell death due to hyperactive apoptosis, such as AIDS, neurodegenerative disease, hematologic diseases, and tissue damage. A further aspect of the present invention relates to the acetylation of BCL6 by inhibiting the deacetylase activity of a protein in the Sir2 class. Doing so prevents expression of differentiation genes in B-cell non-Hodgkin lymphoma (B-NHL) and diffused large B-cell lymphomas (DLBCL). Therefore, inhibiting the NAD+ dependent deacetylase activity of a protein in the Sir2 family of proteins leads to the activation of p53 and either growth or arrest of apoptosis, it is possible to treat various cancers and disease states that are well-known to one of skill in the art.

The method of screening can be used to identify compounds that modulate, e.g., increase or decrease, cell growth, modulate, e.g., slow or speed, aging, modulate, e.g., increase or decrease, lifespan, modulate cellular metabolism, e.g., by increasing or decreasing a metabolic function or rate.

The present invention also relates to a method of modulating the growth of a cell in vivo or in vitro by modulating the Sir2-mediated deacetylation of a transcription factor in the cell.

The compounds identified by the methods of the invention can be used, for example, to treat cancer (e.g., a compound which decreases Sir2-mediated deacetylation of p53) or prevent p53-mediated apoptosis (e.g., a compound which increases Sir2-mediated deacetylation of p53). The compounds can be used in methods of treating a cell or an organism, e.g., a cell or organism that has been exposed to DNA-damaging ionizing radiation, by modulating Sir2 activity in the cell. In the method of treating cancer in a mammal, Sir2 activity can be reduced. In a preferred embodiment, Sir2 activity is reduced by nicotinamide or a nicotinamide analog.

In another embodiment, the invention includes a method of treating a cell that has been exposed to ionizing radiation, the method comprising modulating Sir2 activity in the cell. In a particular embodiment, in a cell which has undergone DNA damage or oxidative stress, Sir2 activity can be modulated to reduce Sir2 activity (e.g., by transfecting a cell with a dominant negative regulatory gene, or by addition or expression of nicotinamide or a nicotinamide analog) which can result in the arrest of the growth cycle of the cell, allowing the cell to repair at least a portion of the DNA damage caused by the ionizing radiation. Once the cell has repaired a portion of the DNA damage, the reduction in Sir2 activity can be removed and the cell cycle of the cell resumed.

The compounds or NAD analogs identified by the methods of the invention can be used in the treatment of diseases or conditions such as cancer, or following DNA damage or oxidative stress. The compounds or NAD analogs can be administered alone or as mixtures with conventional excipients, such as pharmaceutically, or physiologically, acceptable organic, or inorganic carrier substances such as water, salt solutions (e.g., Ringer's solution), alcohols, oils and gelatins. Such preparations can be sterilized and, if desired, mixed with lubricants, preservatives, stabilizers, wetting agents, emulsifiers, salts for influencing osmotic pressure, buffers, coloring, and/or aromatic substances and the like which do not deleteriously react with the NAD analogs or compounds identified by the methods of the invention.

The dosage and frequency (single or multiple doses) of the compound or NAD analog administered to a mammal can vary depending upon a variety of factors, including the duration of DNA damage, oxidative stress or cancer condition.

In some embodiments of the present invention, the rate of aging of a cell, e.g., a yeast cell, invertebrate cell (e.g., fly cell), or vertebrate cell (e.g., mammalian cell, e.g., human or mouse cell) is determined. For example, the rate of aging of the cell can be evaluated by measuring the expression of one or more genes or proteins (e.g., genes or proteins that have an age-related expression pattern), by measuring the cell's resistance to stress, e.g., genotoxic stress or oxidative stress, by measuring one or more metabolic parameters (e.g., protein synthesis or degradation, ubiquinone biosynthesis, cholesterol biosynthesis, ATP levels within the cell, glucose metabolism, nucleic acid metabolism, ribosomal translation rates, etc.), by measuring cellular proliferation, or any combination of measurements thereof.

In other embodiments, the rate of aging of an organism, e.g., an invertebrate (e.g., a worm or a fly) or a vertebrate (e.g., a rodent, e.g., a mouse) is determined. The rate of aging of an organism can be determined by directly measuring the average life span of a group of animals (e.g., a group of genetically matched animals) and comparing the resulting average to the average life span of a control group of animals (e.g., a group of animals that did not receive the test compound but are genetically matched to the group of animals that did receive the test compound). Alternatively, the rate of aging of an organism can be determined visually, e.g., by looking for visible signs of age (e.g., physical appearance or behavior), by measuring the expression of one or more genes or proteins (e.g., genes or proteins that have an age-related expression pattern), by measuring the cell's resistance to genotoxic (e.g., caused by exposure to etoposide, UV irradiation, mutagens, etc.) or oxidative stress, by measuring one or more metabolic parameters (e.g., protein synthesis or degradation, ubiquinone biosynthesis, cholesterol biosynthesis, ATP levels, glucose metabolism, nucleic acid metabolism, ribosomal translation rates, etc.), by measuring cellular proliferation (e.g., of retinal cells, bone cells, white blood cells, etc.), or any combination of measurements thereof. In one embodiment, the visual assessment is for evidence of apoptosis, e.g., nuclear fragmentation.

All animals typically go through a period of growth and maturation followed by a period of progressive and irreversible physiological decline ending in death. The length of time from birth to death is known as the life span of an organism, and each organism has a characteristic average life span. Aging is a physical manifestation of the changes underlying the passage of time as measured by percent of average life span.

In some cases, characteristics of aging can be quite obvious. For example, characteristics of older humans include skin wrinkling, graying of the hair, baldness, and cataracts, as well as hypermelanosis, osteoporosis, cerebral cortical atrophy, lymphoid depletion, thymic atrophy, increased incidence of diabetes type II, atherosclerosis, cancer, and heart disease. Nehlin et al. (2000), Annals NY Acad Sci 980:176-79. Other aspects of mammalian aging include weight loss, lordokyphosis (hunchback spine), absence of vigor, lymphoid atrophy, decreased bone density, dermal thickening and subcutaneous adipose tissue, decreased ability to tolerate stress (including heat or cold, wounding, anesthesia, and hematopoietic precursor cell ablation), liver pathology, atrophy of intestinal villi, skin ulceration, amyloid deposits, and joint diseases. Tyner et al. (2002), Nature 415:45-53.

Those skilled in the art will recognize that the aging process is also manifested at the cellular level, as well as in mitochondria. Cellular aging is manifested in loss of doubling capacity, increased levels of apoptosis, changes in differentiated phenotype, and changes in metabolism, e.g., decreased levels of protein synthesis and turnover.

Given the programmed nature of cellular and organismal aging, it is possible to evaluate the “biological age” of a cell or organism by means of phenotypic characteristics that are correlated with aging. For example, biological age can be deduced from patterns of gene expression, resistance to stress (e.g., oxidative or genotoxic stress), rate of cellular proliferation, and the metabolic characteristics of cells (e.g., rates of protein synthesis and turnover, mitochondrial function, ubiquinone biosynthesis, cholesterol biosynthesis, ATP levels within the cell, levels of a Krebs cycle intermediate in the cell, glucose metabolism, nucleic acid metabolism, ribosomal translation rates, etc.). As used herein, “biological age” is a measure of the age of a cell or organism based upon the molecular characteristics of the cell or organism. Biological age is distinct from “temporal age,” which refers to the age of a cell or organism as measured by days, months, and years.

Further, patients may be treated by gene replacement therapy. One or more copies of a normal target gene, or a portion of the gene that directs the production of a normal target gene protein with target gene function, may be inserted into cells using vectors that include, but are not limited to adenovirus, adenoma-associated virus, and retrovirus vectors, in addition to other particles that introduce DNA into cells, such as liposomes. Additionally, techniques such as those described above may be utilized for the introduction of normal target gene sequences into human cells.

Cells, preferably autologous cells, containing and expressing normal target gene sequences may then be introduced or reintroduced into the patient at positions which allow for the amelioration of metabolic disease symptoms. Such cell replacement techniques may be preferred, for example, when the target gene product is a secreted, extracellular gene product.

In instances where the target gene protein is extracellular, or is a transmembrane protein, any of the administration techniques described, below which are appropriate for peptide administration may be utilized to effectively administer inhibitory target gene antibodies to their site of action.

The identified compounds that inhibit target gene expression, synthesis and/or activity can be administered to a patient at therapeutically effective doses to treat or ameliorate or delay the symptoms of aging. A therapeutically effective dose refers to that amount of the compound sufficient to result in amelioration or delay of symptoms of aging.

Histone deacetylation alters local chromatin structure and consequently can regulate the transcription of a gene in that vicinity. Sir2 proteins can bind to a number of other proteins, termed “Sir2-binding partners.” For example, hSIRT1 binds to p53. In many instances the Sir-2 binding partners are transcription factors, e.g., proteins that recognize specific DNA sites. Interaction between Sir2 and Sir2-binding partners delivers Sir2 to specific regions of a genome and can result in local modification of substrates, e.g., histones and transcription factors localized to the specific region. Accordingly, cellular processes can be regulated by compounds that alter (e.g., enhance or diminish) the ability of a Sir2 protein to interact with a Sir2-binding partner or that alter that ability of a Sir2 protein to modify a substrate. While not wishing to be bound by theory, a Sir2-transcription factor complex may be directed to a region of DNA with a transcription factor binding site; once there, Sir2 may alter the acetylation status of the region, e.g., by deacetylating histones, non-histone proteins, and/or DNA. This would locally raise the concentration of Sir2 and may potentially result in the Sir2-mediated silencing of genes located at or near transcription-factor binding sites. Certain organismal programs such as aging or metabolism and disorders such as cancer can be controlled using such compounds.

While not wishing to be bound by theory, in mammalian cells, signals indicating the successful completion of DNA repair may be relayed via hSir2 to acetylated proteins like p53 that have been charged with the task of imposing a growth arrest following DNA damage. These signals enable hSir2 to reverse part or all of the damage-induced activation of p53 as a transcription factor by deacetylating the K382 residue of p53. By doing so, hSir2 reduces the likelihood of subsequent apoptosis and, at the same time, makes it possible for cells to re-enter the active cell cycle, enabling them to return to the physiological state that they enjoyed prior to sustaining damage to their genomes.

Inactivation of the p53 signaling pathway is involved in the pathogenesis of most if not all human tumors (Hollstein et al., 1994; Lohrum and Vousden, 1999). In about half of these tumors, mutation of the p53 gene itself suffices to derail function. In some of the remaining tumors, loss of p14.sup.ARF, which acts to down-regulate p53 protein levels, has been implicated (Lohrum and Vousden, 1999; Prives and Hall, 1999).

### Mechanism of Sir2

Sirtuin inhibition by nicotinamide has emerged as an important regulatory mechanism of sirtuin activity in vitro and in vivo. Budding yeast grown in the presence of added nicotinamide have defects in Sir2-mediated transcriptional silencing, increased rDNA recombination and a significantly shorter lifespan (Bitterman et al., 2002). Depletion of nicotinamide by PNC1, a yeast enzyme that converts nicotinamide into nicotinic acid, is sufficient to activate Sir2 to extend longevity and prevent nicotinamide-induced inhibition of telomeric and rDNA silencing (Anderson et al., 2003; Gallo et al., 2004). Nicotinamide can inhibit p53 deacetylation by Sir2 cc upon DNA damage in mouse embryonic fibroblast cells (Luo et al., 2001). In human embryonic kidney cells, nicotinamide inhibits the deacetylation of histones H3 and H4 by SirT1, which leads to the loss of transcriptional repression mediated by COUP transcription factor-interacting proteins (Senawong et al., 2003). Interestingly, the sensitivity of yeast Sir2 to nicotinamide differs when it binds to Sir4 or when it is part of the RENT complex (Tanny et al., 2004), suggesting that different cellular partners can further modulate the inhibition of sirtuins by nicotinamide.

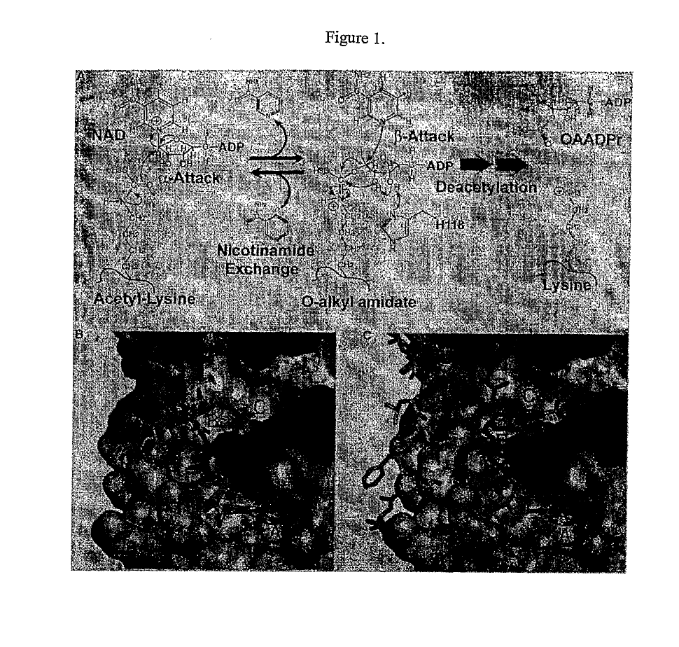

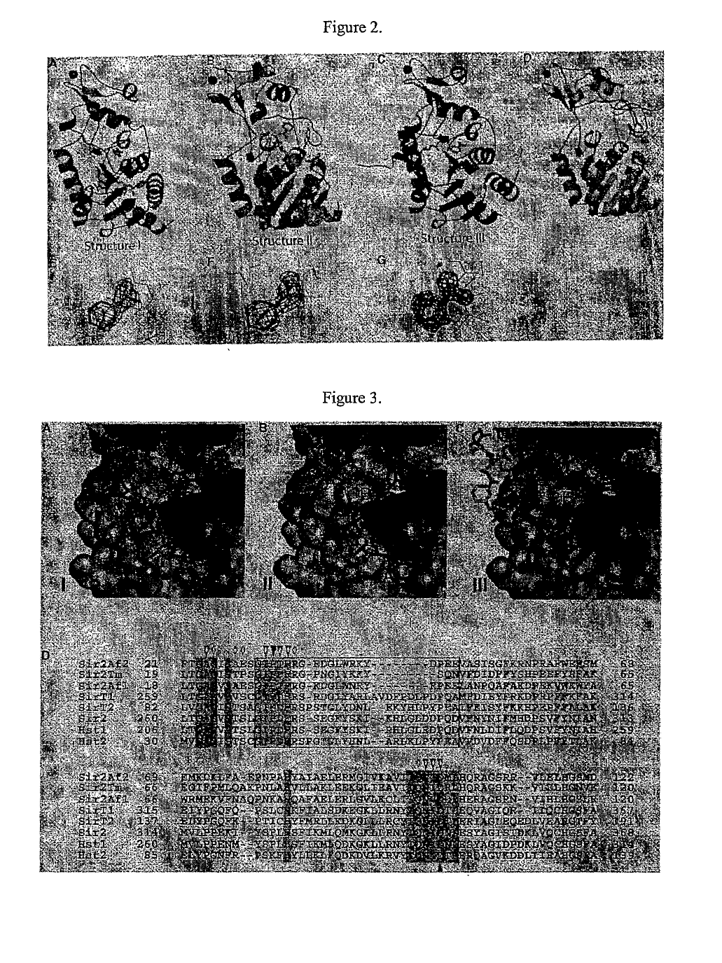

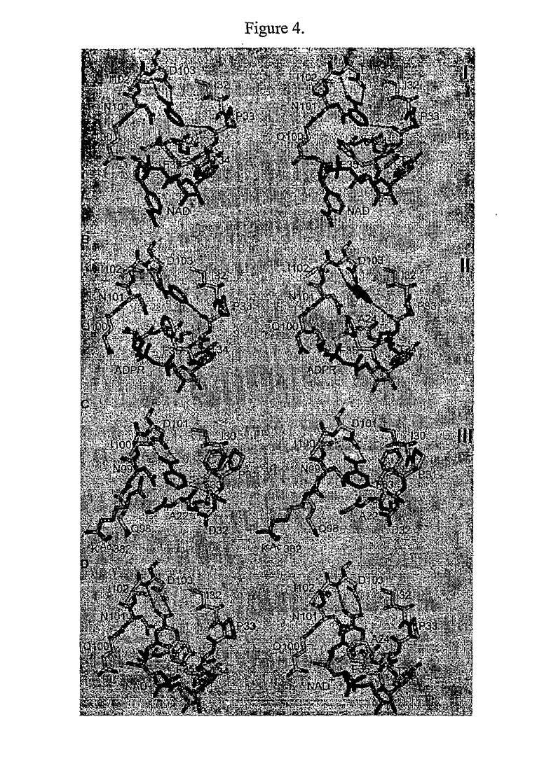

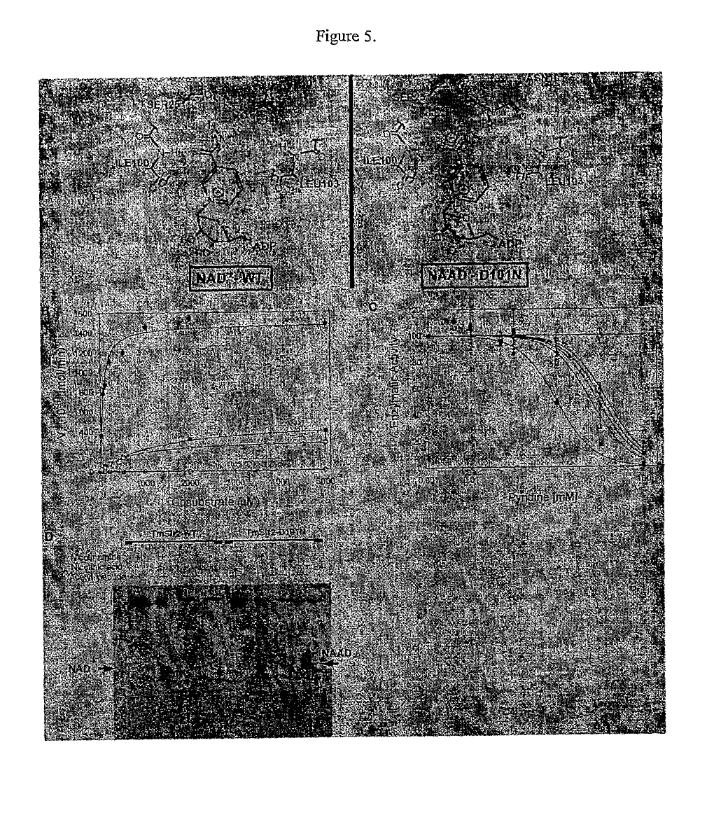

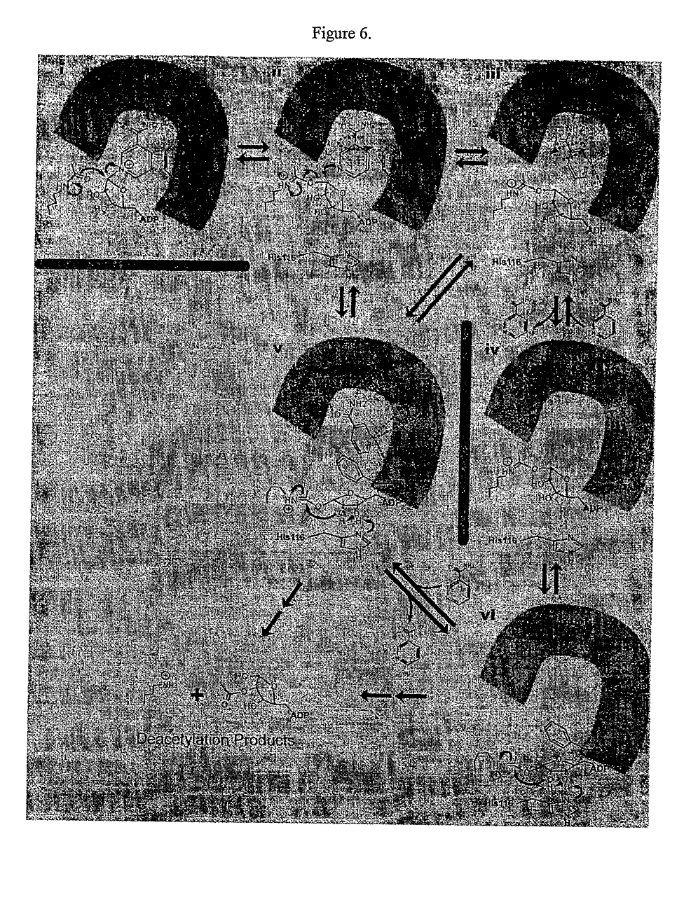

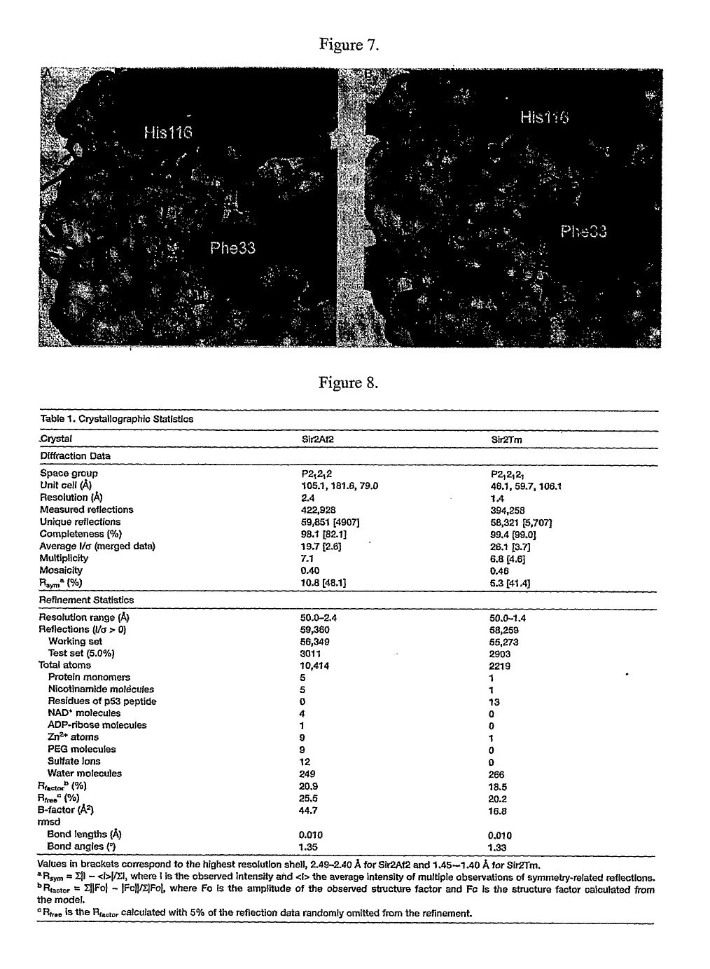

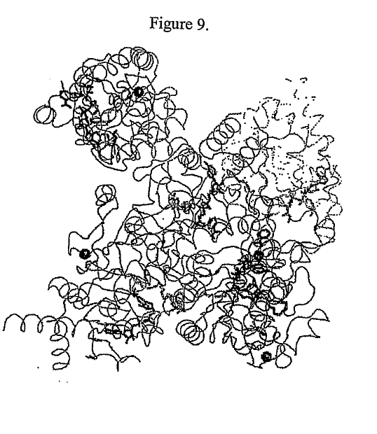

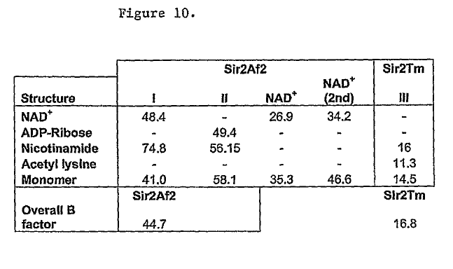

Nicotinamide inhibits the deacetylation activity of sirtuins by reacting with a reaction intermediate. The NAD⁺-dependent deacetylation carried out by sirtuins is thought to begin with a nucleophilic attack of the carbonyl oxygen of acetyl-lysine on the C1′ of the nicotinamide ribose (N-ribose) of NAD⁺, which results in release of nicotinamide and formation of a positively charged O-alkyl-amidate intermediate (Denu, 2003; Sauve et al., 2001; Sauve and Schramm, 2004) (FIG. 1A). Subsequent steps in the reaction lead to the production of OAADPr and deacetylated lysine. If, however, nicotinamide binds to the enzyme when it contains the O-alkyl-amidate intermediate, nicotinamide can react with the intermediate in a process known as nicotinamide exchange, in which NAD⁺ and acetyl lysine are re-formed (Jackson et al., 2003; Sauve et al., 2001; Sauve and Schramm, 2003) (FIG. 1A). High concentrations of nicotinamide increase the rate of the nicotinamide exchange reaction, which occurs at the expense of the deacetylation activity. Crystal structures of sirtuins have shown that NAD⁺ can bind in various “non-productive” conformations that are not suitable for catalysis (Avalos et al., 2004; Min et al., 2001) (FIG. 1B). However, simultaneous binding of NAD⁺ and substrate peptide to the enzyme promotes binding of NAD⁺ in a distinct “productive” conformation that places the nicotinamide ring in a highly conserved pocket, called the C pocket, where it is activated for catalysis (Avalos et al., 2004; Zhao et al., 2004) (FIG. 1C). It has been a matter of controversy as to whether the C pocket is also used in the exchange reaction that results in nicotinamide inhibition (Avalos et al., 2004; Bitterman et al., 2002), or if an alternative pocket serves as a nicotinamide-binding site for this purpose (Zhao et al., 2004). The two models have different implications for the mechanisms of NAD⁺ cleavage, nicotinamide exchange and the regulation of sirtuins by nicotinamide.

In order to address the structural basis for the enzymatic mechanism by which nicotinamide regulates the deacetylation activity of sirtuins, we have determined crystal structures of sirtuins bound to nicotinamide and used the findings to engineer an altered specificity enzyme that can catalyze NAAD-dependent deacetylation and that is inhibited by nicotinic acid. The structures of archaeal Sir2Af2 and bacterial Sir2Tm reported here show that free nicotinamide binds in the same conserved C pocket in which NAD⁺ is activated for catalysis, supporting a dual role for the C pocket in both nicotinamide exchange and deacetylation. In order to test whether the C pocket is indeed responsible for NAD⁺ cleavage and nicotinamide exchange, we engineered a single point mutation in the C pocket that was designed to enable the enzyme to use nicotinic acid adenine dinucleotide (NAAD), in place of NAD⁺ to deacetylate lysine residues. The mutant acquired the predicted NAAD-dependent deacetylation activity while retaining some NAD⁺-dependent activity. Importantly, the mutant lost sensitivity to nicotinamide inhibition while acquiring sensitivity to nicotinic acid inhibition and the ability to catalyze nicotinic acid exchange. The results of these biochemical and structural studies allow us to propose a structure-based mechanism for the non-competitive inhibition and regulation of sirtuins by nicotinamide, and shed light on the mechanism of NAD⁺ cleavage by sirtuins and their cosubstrate.

We have determined structures of archaeal and bacterial sirtuins bound to nicotinamide. Three independent structures of Archaeoglobus fulgidus Sir2Af2 bound to nicotinamide were determined from a single crystal diffracting to 2.4 Å resolution. The crystal was grown in the presence of NAD⁺, PEG400 and nicotinamide and was isomorphous to crystals grown in the absence of nicotinamide that were reported in a previous study (Avalos et al., 2004). The crystals contain five crystallographically independent monomers in the asymmetric unit that are in differently liganded states. Two of the five monomers in the asymmetric unit are ternary complexes containing nicotinamide bound in the C pocket of the active site of Sir2Af2. One of these structures (which we shall call “Structure I”) is also bound to NAD⁺ in a non-productive conformation (FIGS. 2A and 3A). Another ternary complex (which we call “Structure II”) also contains α-ADP-ribose bound in the active site (FIGS. 2B and 3B). The nicotinamide in structure II is well-ordered, while that in structure I appears to be present at somewhat lower occupancy (FIG. 10). The density corresponding to the carboxamide and part of the pyrole ring, along with the assumption that the molecule is planar, was used to position the structure I nicotinamide in the map. A third complex in the crystal, which contains NAD⁺ bound in a non-productive conformation has density suggestive of nicotinamide bound at low occupancy and is therefore not used in our analysis. The remaining two monomers in the asymmetric unit are bound to NAD⁺ in a productive conformation that occupies the C pocket, and to a PEG molecule that lies in the acetyl-lysine-binding tunnel. These two complexes are virtually identical to one another, as well as to the previously reported structure of these complexes determined from crystals grown in the absence of nicotinamide (Avalos et al., 2004). Two additional nicotinamide molecules mediate apparently non-specific interactions between monomers in the asymmetric unit that are far from the active site.

The 1.4 Å structure of Sir2Tm, from the thermophilic bacterium Thermotoga maritima, was determined in complex with nicotinamide and an acetylated p53 peptide, which is an in vitro substrate for Sir2Tm (Structure III, FIG. 2C). A single well-ordered nicotinamide molecule is bound to the C pocket of Sir2Tm (FIGS. 2C and 3C). The peptide and acetylated lysine bind to the enzyme in a manner similar to that observed in the structure of Sir2Af2 bound to the same peptide (Avalos et al., 2002). We shall refer to the numbering of sirtuins residues according to the Sir2Af2 protein, except where otherwise noted (FIG. 3D).

Previous studies (Avalos et al., 2002; Finnin et al., 2001; Min et al., 2001) have shown that sirtuins contain a highly flexible region, called the flexible loop, which adopts a variety of conformations in different crystal structures and is in some cases partially disordered (FIG. 2). This 15-30 amino acid flexible loop includes some of the most highly conserved residues in sirtuins, which form the front wall of the C pocket (FIG. 3D). Structures I and II of Sir2Af2 bound to nicotinamide have ordered flexible loops that adopt different conformations (FIGS. 2A and B). In contrast, the structure of Sir2Tm bound to nicotinamide (Structure III) has a partially disordered flexible loop from Arg34 to Ser44 (FIG. 2C), but still shows strong electron density for the conserved Phe33 that forms part of the C pocket (FIG. 3C). These structures suggest that the flexible loop is influenced by NAD⁺ binding, which can trigger the assembly and disassembly of the C pocket.

The structures presented here show that nicotinamide can bind to sirtuins simultaneously with peptide, ADP ribose, or NAD⁺ that is in a non-productive conformation. These ternary structures show that nicotinamide can bind in a collection of alternative positions that are anchored by the carboxamide group, but leave the pyridine ring free to pivot inside the C pocket. The C pocket is a largely hydrophobic cavity that contains the most highly conserved residues in the catalytic core of sirtuins (FIG. 3D). In all structures reported in this study, the carboxamide group of nicotinamide forms a conserved set of interactions that anchor the nicotinamide in the C pocket. The carboxamide amino of nicotinamide interacts with the side chain of a conserved aspartic acid in the C pocket (Asp103), while the carboxamide oxygen interacts with the backbone amino group of a conserved isoleucine (Ile102) (FIG. 4). Additional conserved interactions with the carboxamide include van der Waals contacts with the side chains of Ile102 and Asn 101, which is highly conserved. By contrast, the pyridine ring of nicotinamide can adopt a variety of conformations (FIG. 4), as reflected in the different positions of the ring in complexes II and III and as suggested by the weaker electron density for the ring in structure I (FIG. 2E). This variability is due not only to differences in the positioning of the pyridine ring inside the pocket, but also to variations in the conformation of the flexible wall of the C pocket itself (FIG. 2D). The differences in the C pocket flexible wall and hence the variability of its interactions with nicotinamide are due at least in part to variations in the conformation of the flexible loop. Consequently, the nicotinamide can make alternative interactions with the highly conserved residues Ala 24, Ile 32, Phe 35 and Ile 102 (FIG. 4A-C). Phe 35 is of particular interest, since it displays the largest conformational differences within the C pocket and the greatest diversity of interactions with the nicotinamide (FIG. 4). As discussed below, this side chain appears to play an important role in the catalytic mechanism.

The interactions of free nicotinamide with the C pocket have some similarities with those of the nicotinamide moiety of NAD⁺, but also differ in important ways. In the structure of Sir2Af2 bound to productive NAD⁺ (FIGS. 1C and 4D) (see also (Avalos et al., 2004)), the interactions of the carboxamide group of nicotinamide with Asp103, Ile102 and Asn101 are very similar to those formed by free nicotinamide (FIG. 4A-C). This similar interaction occurs even though the nicotinamide moiety, when part of NAD⁺, is constrained by its glycosidic bond with the C1′ of the N-ribose (FIG. 4D), which leads to a rotation of the NAD⁺ carboxamide by approximately 150° from its most favorable conformation (Bell et al., 1997; Olsen et al., 2003). In contrast, the ring in free nicotinamide can adopt the most favored conformation, with the carboxamide nearly coplanar with the nicotinamide ring (Olsen et al., 2003). The N1 and C5 of nicotinamide cannot be distinguished in the electron density map, and the two possible low-energy rotamers are probably in equilibrium. However, when the N1 of nicotinamide is in the cis position with the carboxamide amino (N1-NH₂-Cis rotamer), the N1 can form favorable contacts with conserved residues in the C pocket that could shift the equilibrium towards this rotamer. The N1-NH₂-Cis rotamer, which is opposite to the rotamer in the productive NAD⁺ complex (FIG. 4D), places the nicotinamide N1 3.3 Å from the backbone oxygen of the conserved Pro33 in structure II (FIG. 4A), with a similar distance predicted for the less well-determined structure I (3.5 Å, FIG. 4B). In structure III, the N1 of nicotinamide in this rotamer is 3.1 Å from the amino backbone of Phe33 (Sir2Tm numbering) and 3.5 Å from the backbone oxygen of Pro31 (FIG. 4C). The alternative nicotinamide rotamer (N1-NH2-trans) does not permit the N1 to form significant interactions with the enzyme in any of the complexes. This suggests that, upon cleavage of NAD⁺, the pyridine ring of the nicotinamide may flip about its carboxamide group to relieve the stress induced on the NAD⁺ and form new interactions between the N1 of nicotinamide and residues deep inside the C pocket.

Our structural studies suggest that the C pocket is the sole binding site for free nicotinamide and thus the regulatory site for nicotinamide inhibition. To test the relevance of the observed interactions with nicotinamide to the NAD⁺-dependent deacetylation and nicotinamide exchange activities of sirtuins, we engineered a point mutant in the C pocket designed to alter the enzyme's cosubstrate specificity. Based on our structures, we reasoned that a mutation of the conserved aspartic acid in the C pocket (Asp101 in Sir2Tm) to asparagine would confer on the mutant an NAAD-dependent deacetylation activity, since the amino group of asparagine could hydrogen-bond with the carboxylate of NAAD just as the wild type aspartic acid hydrogen bonds with the amino group of NAD⁺ (FIG. 5A). If the C pocket is important not only for NAD⁺ cleavage but also for nicotinamide inhibition, this mutation should also lead to a loss of sensitivity to nicotinamide inhibition and a gain of sensitivity to nicotinic acid inhibition due to an acquired ability to catalyze nicotinic acid exchange.

As expected, the Sir2Tm enzyme containing an Asp101 to Asn substitution (D101N) exhibits a significant loss in NAD⁺-dependent deacetylation activity. The Sir2Tm-D101N mutant has significantly reduced catalytic power, with an apparent k_{cat} of (1.8±0.1)×10⁻³ s⁻¹ two orders of magnitude lower than the wild type k_{cat} of 0.170±0.006 s⁻¹ (FIG. 5B). In addition, the apparent K_{M} for NAD⁺ of the mutant enzyme, 1.17±0.18 mM, represents a 22-fold increase from the 53±11 μM K_{M} value of the wild type Sir2Tm (FIG. 5B). A significant loss in NAD⁺-dependent deacetylation activity was also found when the analogous substitution was introduced into Sir2Af2.

To test the effect of the C-pocket aspartic acid mutation on co-substrate specificity, we assayed the ability of the Sir2Tm-D101N mutant to deacetylate a p53-derived peptide using NAAD as a cosubstrate instead of NAD⁺. As predicted, mutation of the conserved aspartic acid in the C pocket to asparagine enables the mutant enzyme to carry out NAAD-dependent deacetylation, whereas the wild type enzyme exhibits no detectable deacetylation activity with NAAD as a cosubstrate (FIG. 5B). The acquired NAAD-dependent deacetylation activity of the Sir2Tm-D101N mutant, with an apparent k_{cat} of (1.1±0.1)×10⁻³ s⁻¹ is comparable to the mutant enzyme's NAD⁺-dependent activity. The mutant has an apparent K_{M} for NAAD of 617±43 μM, which is approximately half its K_{M} for NAD⁺ (FIG. 5B). Furthermore, the apparent second order rate constant of the Sir2TmD101N mutant (k_{cat}/K_{M}) for NAD⁺ of (1.5±0.4)×10⁻³ s⁻¹ mM⁻¹ is comparable to its apparent k_{cat}/K_{M} for NAAD of (1.8±0.3)×10⁻³ s⁻¹ mM⁻¹, and they are three orders of magnitude lower than the wild type apparent k_{cat}/K_{M} for NAD⁺ of 3.2±0.8 s⁻¹ mM⁻¹. The D101N point mutation in Sir2Tm therefore results not only in a significant drop in catalytic power and loss of specificity for NAD⁺, but also in the loss of cosubstrate selectivity, as the mutant appears to be unable to discriminate between NAD⁺ and NAAD.

Mutation of the aspartic acid in the C pocket also alters the sensitivity of the enzyme to inhibition by nicotinamide and nicotinic acid. As predicted, the Sir2Tm-D101N mutant has significantly reduced sensitivity to nicotinamide inhibition. In fluorescence-based assays monitoring deacetylation activity, the mutation causes the IC₅₀ of nicotinamide to increase an order of magnitude, from 1.0±0.2 mM in the wild type to 9.0±2.0 mM in the Sir2Tm-D101N mutant (FIG. 5C). In addition, the NAD⁺-dependent deacetylation activity of the Sir2Tm-D101N mutant can be inhibited by nicotinic acid, with an IC₅₀ of 11.3±3.3 mM, whereas nicotinic acid added to concentrations of up to 100 mM fail to inhibit the wild type enzyme (FIG. 5C). Importantly, the acquired NAAD-dependent deacetylation activity of the Sir2Tm-D101N mutant is also inhibited by nicotinic acid, as well as nicotinamide, with IC₅₀ values of 6.2±2.0 mM and 14.6±3.4 mM, respectively (FIG. 5C). The nicotinamide and nicotinic acid IC₅₀ values for both the NAD⁺- and NAAD-dependent activities of the Sir2Tm-D101N mutant are similar, suggesting that the mutant has not only lost sensitivity to nicotinamide, but also its ability to discriminate between nicotinamide and nicotinic acid (FIG. 5C). Similar results were obtained when deacetylation activity was assayed by monitoring NAD⁺ consumption.

To assay directly the nicotinamide and nicotinic acid exchange activities of the wild type and mutant Sir2Tm, we incubated the enzymes with unlabelled NAD⁺ and acetylated peptide in the presence of ¹⁴C-labelled nicotinamide or nicotinic acid and separated the products using thin layer chromatography (FIG. 5D). As expected from the inhibition experiments, both enzymes could catalyze formation of labeled NAD⁺ through the nicotinamide exchange activity. Most remarkably, the Sir2Tm-D101N mutant is able to synthesize ¹⁴C-labelled NAAD by using the base exchange reaction to catalyze replacement of the unlabelled nicotinamide ring of NAD⁺ with ¹⁴C-labelled nicotinic acid, which the wild type enzyme cannot do. This striking new enzymatic activity must be the consequence of a nicotinic acid exchange activity conferred by the D101N mutation, consistent with its acquired NAAD-dependent deacetylation activity and sensitivity to nicotinic acid inhibition.

We have shown that sirtuins contain a multifunctional site that is directly involved in NAD⁺ cleavage, base exchange activity and nicotinamide regulation. This conclusion rests on our crystallographic studies showing free nicotinamide bound in this site, known as the C pocket (Min et al., 2001), and on our ability to introduce a point mutation in the C pocket that alters the cosubstrate specificity and inhibitor sensitivity of the enzyme. Our striking finding that a single Asp->Asn change in the C pocket enables the enzyme to catalyze NAAD-dependent deacetylation, be inhibited by nicotinic acid, and synthesize NAAD from NAD⁺ and nicotinic acid strongly supports the role of the C pocket as the sole nicotinamide binding site in sirtuins. We had previously shown that the same C pocket binds the nicotinamide moiety of NAD⁺ when this cosubstrate binds to the enzyme in a productive conformation that is poised for catalysis (Avalos et al., 2004). Our findings therefore argue against the presence of a second regulatory binding site for nicotinamide, as has been proposed (Zhao et al., 2004). The details of the interactions of sirtuins with both NAD⁺ and free nicotinamide provide a mechanism for regulating the enzyme's activity in response to these molecules.

Our results highlight the role the C pocket and the conserved aspartic acid it contains in conferring the specificity for both NAD⁺ and nicotinamide that are critical for sirtuin function in vivo. Sirtuins have very high specificity for NAD⁺, as relatively small changes in the nicotinamide ring of NAD⁺ result in the most dramatic losses of binding affinity and reactivity, as is the case for NADH and NAAD (Schmidt et al., 2004). Similarly, the fine specificity of sirtuins for nicotinamide in the base exchange reaction makes them insensitive to inhibition by other metabolites, especially nicotinic acid (Schmidt et al., 2004). The latter property is particularly relevant to the role of the yeast PNC1 enzyme in transcriptional silencing and replicative lifespan, where it is believed to relieve inhibition of Sir2 by converting nicotinamide into nicotinic acid (Anderson et al., 2002; Anderson et al., 2003; Sandmeier et al., 2002). The ability of sirtuins to discriminate between NAD⁺ and NAAD as cosubstrate, as well as between nicotinamide and nicotinic acid as inhibitors, is therefore crucial for the activity and regulation of sirtuins in the cell.

The structures of Sir2Af2 and Sir2Tm with bound nicotinamide reveal how sirtuins sequester the nicotinamide that is cleaved from NAD⁺ in the initial step of catalysis, reducing base exchange and thus promoting deacetylation. The enzymatic reaction begins with cleavage of nicotinamide from NAD⁺ and formation of a reactive O-alkyl amidate intermediate (Sauve et al., 2001) (FIG. 1A). Since the fast rate of nicotinamide condensation with the O-alkyl amidate intermediate is similar to the rate of NAD⁺ cleavage (Jackson et al., 2003), the enzyme needs to release the cleaved nicotinamide quickly or sequester it in order to attenuate the immediate reversal of NAD⁺ cleavage, as has been proposed for ADP-ribosyltransferases (Han et al., 1999). A comparison of complexes containing cleaved nicotinamide with that containing productive NAD⁺ suggests how the enzyme could entrap the cleaved nicotinamide. Although a common set of hydrogen bonding interactions with the carboxamide always anchors nicotinamide in the C pocket, free nicotinamide binds in a low-energy conformation in which the carboxamide and pyridine ring are almost coplanar, while a less favorable out-of-plane rotamer is found in the productive complex with NAD⁺ (Avalos et al., 2004; Zhao et al., 2004). This suggests that the positively charged nicotinamide moiety of NAD⁺ shifts from a distorted, high-energy conformation to a collection of relaxed, low-energy states upon NAD⁺ cleavage. The enzyme may simply use the energy gained from the release of strain upon NAD⁺ cleavage to allow the pyridine ring of nicotinamide to rotate—or “flip”—along the carboxamide group dihedral until it adopts its most stable rotamer, allowing the N1 of nicotinamide to make favorable interactions with conserved residues in the C pocket.

Our new findings allow us to propose a structure-based mechanism for the regulation of sirtuins by nicotinamide (FIG. 6). The initial steps in the NAD⁺ dependent deacetylation reaction lead to formation of the O-alkyl amidate intermediate and free nicotinamide, which has been cleaved from NAD⁺ (FIG. 6i, ii). The pyridine ring of the cleaved nicotinamide can flip to more favorable conformations, in which the pyridine ring is free to adopt a variety of positions that allow its N1 to interact with residues inside the C pocket, while the carboxamide remains anchored through hydrogen bond interactions (FIGS. 4 and 6iii). The bound nicotinamide can exist in either an entrapped or a reactive state that are interchangeable through a flipping mechanism (FIGS. 6ii and iii). In the entrapped state, the nicotinamide places the N1 on the distal side of the C pocket, preventing reaction with the O-alkyl-amidate intermediate and allowing the deacetylation reaction to proceed (FIG. 6iii). If the pyridine ring flips about its carboxamide group, the N1 of nicotinamide can move into a position to react with the C1′ of the alkyl-amidate intermediate, leading to base exchange (FIG. 6ii). Based on crystal structures of sirtuins bound to acetylated peptide (Avalos et al., 2002; Finnin et al., 2001; Min et al., 2001), 2′O-acetyl ADP ribose (Zhao et al., 2003) and NAD⁺ (Avalos et al., 2004) we propose that the alkyl-amidate intermediate exists in two alternative conformations, either contracted or extended (FIG. 7), that could influence whether bound nicotinamide reacts with the intermediate to re-form NAD⁺. The contracted conformation would favor deacetylation, since its C1′ is far from nicotinamide bound in the C pocket and the N-ribose 2′ and 3′ OH groups are near His116 (Sir2Tm numbering), which catalyzes subsequent steps in the deacetylation reaction (Denu, 2003; Sauve et al., 2001; Sauve and Schramm, 2004) (FIGS. 6v and 7A). Furthermore, the O-alkyl amidate intermediate in the contracted conformation is protected from the solvent and nicotinamide bound in the C pocket by Phe33 (FIGS. 6v and 7A). When the intermediate is in the extended conformation, it is further from His116, closer to the C pocket and exposed by Phe33 (FIGS. 6ii and 7B), making it more likely to react with the bound nicotinamide and re-form NAD⁺ and acetyl-lysine. The conformational variability of the flexible loop (FIG. 2E) may allow nicotinamide release by allowing the partial disassembly of the C pocket. Re-binding of nicotinamide can lead to the inhibitory base exchange reaction if the rebinding occurs when the enzyme is bound to the O-alkyl amidate intermediate.

The kinetic significance of the alternative modes of nicotinamide binding remains to be determined. It is formally possible that the energy released by relieving NAD⁺ strain is enough to expel nicotinamide out of the active site completely and that the alternative nicotinamide binding conformations are the consequence of the high nicotinamide concentrations used in the crystallizations. Nevertheless, the observed alternate modes of nicotinamide binding could be used by the enzyme to entrap nicotinamide and attenuate the re-formation of NAD⁺.

If sirtuins contain a single nicotinamide binding site that functions in both deacetylation and base exchange, why is nicotinamide inhibition non-competitive (Bitterman et al., 2002; Jackson et al., 2003; Sauve and Schramm, 2003)? One possible explanation is that the affinity of nicotinamide for the C pocket is very low in the absence of an O-alkyl amidate intermediate, and that we were able to see it in our electron density maps only because the crystals were grown at high nicotinamide concentrations (see Examples). Indeed, there is some evidence that high, non-physiological concentrations of nicotinamide (approaching 0.1 M) may inhibit the deacetylation reaction in a manner that is competitive with NAD⁺.

We had previously suggested (Avalos et al., 2004) that binding of an acetylated peptide to sirtuins promotes binding of NAD+ in a strained conformation required for catalysis, which is supported by the nicotinamide interactions in the structure of Hst2 bound to carba-NAD+ and acetylated peptide (Zhao et al., 2004) and is consistent with recent kinetic studies showing that NAD⁺ binds after acetylated peptide (Borra et al., 2004). Our structures showing that the nicotinamide product binds in the C pocket in conformations that are energetically lower than that of the productive NAD⁺ supports this model of ground state destabilization. However, the increase in K_{M} for cosubstrate observed in the TmSir2-D101N mutant suggests that other factors involving Asp101, probably transition state stabilization, are also important for NAD⁺ cleavage. The 100-fold loss in catalytic power of the mutant may be due to its inability to distort the carboxamide of NAD⁺ or the carboxylate of NAAD. The out-of-plane rotation of the carboxamide could play a role in catalysis by disrupting the electronic resonance between the carboxamide and the pyridine ring, which could alter the electronic distribution on the pyridine ring in a way that weakens the glycosidic bond and promotes NAD⁺ cleavage. It is possible that the NAAD-dependent deacetylation activity of the mutant is not as robust as the wild type enzyme's NAD⁺-dependent activity because of inherent differences in the charge and electronic distribution between the positively charged NAD⁺ and the electronically neutral NAAD.

Our findings regarding the central regulatory role of the C pocket indicate a structural basis for the action of small molecules that either inhibit or stimulate sirtuin activity. Molecules that can prevent NAD⁺ from binding in its productive conformation can act as competitive inhibitors, while those that are able to participate in the flipping mechanism and react with the O-alkyl amidate intermediate can act as non-competitive inhibitors of the sirtuin deacetylation reaction. The latter instance is likely to be the case in the inhibition of Hst2 by thionicotinamide and 3-hydroxypyridine, two nicotinamide analogs that can participate in pyridine base exchange reactions in Hst2 (Jackson et al., 2003). Conversely, molecules that bind in the C pocket while the enzyme is bound to the O-alkyl amidate intermediate but are either inert or unable to flip for activation could stimulate the deacetylation reaction by reducing the inhibitory effect of intracellular nicotinamide. The latter mechanism is consistent with the observed stimulatory effect of isonicotinamide (Sauve and Schramm), which also binds in the C pocket.

The role of the flexible loop in nicotinamide binding and release indicates a mechanism by which protein partners may modulate sirtuin activity. Although the carboxamide group of nicotinamide is anchored to the rigid inner wall of the C pocket, the pyridine ring makes a variety of contacts with residues in the flexible loop, whose conformation is highly variable in the different sirtuin structures. Binding of proteins or small molecules that affect the conformation or mobility of the flexible loop could therefore affect deacetylation activity and nicotinamide exchange. This feature of the protein could therefore be exploited to design or select for proteins or small molecules that increase or decrease sirtuin activity by interacting with the flexible loop. Interestingly, approximately one third of the flexible loop comprising the flexible wall of the C pocket contains some of the most conserved residues in sirtuins, while the remainder of the flexible loop is one of the most variable regions of the catalytic core of sirtuins (FIG. 3D). This peculiarity hints at the possibility that the variable region of the flexible loop participates in interactions that various sirtuins make with different cellular partners, each of which may have distinct consequences for enzyme activity. This hypothesis could explain the differing nicotinamide sensitivity of yeast Sir2 when it forms different protein complexes, as well as the stimulation of Sir2 activity when it is bound to Sir4 (Tanny et al., 2004). It is also possible that the binding of sirtuins to certain substrates could similarly affect the flexible loop and hence enzyme activity. Interestingly, the human SirT1 protein interacts with a variety of cellular proteins (Brunet et al., 2004; Motta et al., 2004; Picard et al., 2004; Takata and Ishikawa, 2003; Vaquero et al., 2004; Vaziri et al., 2001) and is sensitive to nicotinamide in vitro. The various interactions of human sirtuins with their cellular partners have the potential to play an important role in regulating this important class of deacetylase enzymes.

### Pharmaceutical Compositions

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration. The methods of the invention further include administering to a subject a therapeutically effective amount of a compound in combination with another pharmaceutically active compound. Pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference.

The phrase “pharmaceutically acceptable” is refers to those compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, involved in carrying or transporting the subject chemical from one organ, or portion of the body, to another organ, or portion of the body. Each carrier must be “acceptable” in the sense of being compatible with the other ingredients of the formulation and not injurious to the patient.

Methods of preparing these compositions include the step of bringing into association a compound(s) with the carrier and, optionally, one or more accessory ingredients. These compositions may also contain adjuvants such as preservatives, wetting agents, emulsifying agents and dispersing agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. Alternatively, delayed absorption of a parenterally-administered drug form is accomplished by dissolving or suspending the drug in an oil vehicle.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient.

### EXAMPLES

### Example 1

### Protein Expression and Purification

The Sir2Af2 from Archaeoglobus fulgidus and Sir2Tm from Thermotoga maritima, wild type and mutant enzymes, were expressed in E. coli and purified as described previously (Smith et al., 2002). The mutagenesis of Sir2Af2 and Sir2Tm was carried out using Quick Change (Stratagene).

### Example 2

### Crystallization of Sirtuin Complexes with Nicotinamide

Purified Sir2Af2 enzyme was dialyzed into 10 mM HEPES pH 7.4 with 1 mM Tris (2-carboxyethyl)-phosphine TCEP and concentrated to 20 mg/ml. Prior to crystallization trials, 5.5 μL of a neutralized solution of 100 mM NAD⁺ was added to 50 μl of the Sir2Af2 solution, to a final concentration of 10 mM NAD⁺. Crystals were grown by the hanging drop method in 0.1 M HEPES pH 7.4, with 1.8 M ammonium sulfate, 1% PEG400 and 70 mM nicotinamide and formed in space group P2₁2₁2 with unit cell dimensions a=105.1 Å, b=181.6 Å, c=79.0 Å. Crystals were flash-frozen in nujol oil (Plough Inc.) and stored in liquid nitrogen until use.

Purified Sir2Tm enzyme was dialyzed into 10 mM HEPES pH 7.4 and concentrated to 16 mg/ml, and 5 μl of a 40 mM solution of acetylated p53 peptide (372-KKGQSTSRHK-K(Ac)-LMFKTEG-389) was added to a final concentration of 10 mg/mL Sir2Tm and 4 mM peptide. Crystals were grown by the hanging drop method in 100 mM CHES, pH 9.6, with 16% PEG 3350 and 100 mM nicotinamide and formed in space group P2₁2₁2₁ with unit cell dimensions a=46.1 Å, b=59.8 Å, c=106.2 Å. Crystals were flash-frozen in mother liquor containing 20% ethylene glycol and stored in liquid nitrogen until use.

### Example 3

### Structure Determination

Diffraction data on Sir2Af2 crystals were recorded at beamline X25 of the National Synchrotron Light Source (NSLS) with a Quantum CCD detector and reduced with HKL2000 (Otwinowski and Minor, 1997) and CCP4. The crystals are isomorphous to the structure of Sir2Af2 bound to NAD⁺ and ADP ribose (Avalos et al., 2004) (accession number 1S7G), which was used to calculate phases, compute a difference Fourier map with CNS (Brunger et al., 1998) and locate the electron density corresponding to the nicotinamide and additional NAD⁺ molecules. The density corresponding to nicotinamide could not otherwise be accounted for by bound water or ions. The structure was built using Xfit (McRee, 1999) and refined with simulating annealing and energy minimization in CNS (Brunger et al., 1998). The positions of the nicotinamide molecules, as well as flexible regions in the proteins, were verified with simulated annealing omit maps (Brunger et al., 1998) (FIGS. 2E and 2G). The final model contains five Sir2Af2 monomers, four molecules of NAD⁺, one of ADP ribose, five of nicotinamide, nine of PEG, nine zinc atoms, twelve sulfates and 249 waters. The crystallographic statistics are summarized in FIG. 8; Table 1. Values for protein and ligand B factors are shown in supplemental data (FIG. 10).

Diffraction data on Sir2Tm crystals were collected at NSLS beamline X4A, and processed as described above. The structure was solved by molecular replacement with MOLREP (Vagin and Teplyakov, 1997), using as a search model the structure of the Sir2Tm apoenzyme (JLA and CW, unpublished) broken into two segments: the Rossmann domain and the small domain. The structures were built using Xfit (McRee, 1999) and refined with simulating annealing and energy minimization in CNS (Brunger et al., 1998). The position of the nicotinamide was verified with simulated annealing omit maps (FIG. 2G). Residues Arg34-Ser44 of the flexible loop showed no corresponding electron density, indicating that this region is disordered. The final model contains one monomer of Sir2Tm, thirteen residues from the acetylated p53 peptide, one nicotinamide, one zinc atom and 266 waters. Crystallographic statistics are summarized in FIG. 8; Table 1.

### Example 4

### Measurement of Deacetylation Activity Using a Fluorolabeled Peptide

The deacetylation activity was measured using the Fluor de Lys-SirT1 assay (Biomol), using a peptide containing amino acids 379-RHK-K(Ac)-382 of p53 as substrate. The initial rates of the NAD⁺- and NAAD-dependent deacetylation activities of Sir2Tm wild type and D101N mutant enzymes were measured at different concentrations of dinucleotide. The reactions were carried out at 37° C. in a 50 μL reaction volume containing 50 mM Tris, pH 8, 50 mM NaCl and 400 μM fluorolabeled peptide (˜10 times K_{M}). The enzyme concentration of the wild type enzyme was 80 μg/mL and that of the D101N mutant was 2.56 mg/ml. The NAAD-dependent activity of the wild type enzyme was undetectable even at enzyme concentration of 2.56 mg/ml and incubation periods of 4 hours. Reactions were done at least in triplicate. The data were fitted to the Michaelis-Menten equation using SigmaPlot to obtain the kinetic constants. This assay was also used to measure the inhibition by nicotinamide and nicotinic acid, using 500 μM NAD⁺ for the wild type enzyme and 2 mM of NAD⁺ or NAAD for the mutant. The initial rates were measured at different concentrations of nicotinamide and nicotinic acid, and the reaction conditions were the same as above, except the D101N mutant enzyme was used at 1.6 mg/ml. The data were fitted to equation (1) using SigmaPlot (SYSTAT) to calculate the IC₅₀ values:

v_{I}=v₀(1−(I/(IC₅₀+I))) Equation (1)

where v₀ is the initial rate of the uninhibited reaction and v_{I} is the initial rate of the reaction at concentration I of inhibitor.

### Example 5

### Thin Layer Chromatography (TLC) Detection of Base Exchange Activities

The nicotinamide and nicotinic acid exchange reactions were carried out in 20 μL containing 50 mM sodium phosphate, pH 8.0, 0.5 mM DTT, 2 mM NAD⁺ and 500 μM of the acetylated p53 peptide used in the crystallographic studies and NAD⁺ consumption assay. The reactions contained 0.1 mM of ¹⁴C-nicotinamide (Moravek Biochemicals MC1427, 53 mCi/mmol) or ¹⁴C-nicotinic acid (Moravek Biochemicals MC1324, 54 mCi/mmol), using 80 μg/mL of the wild type enzyme or 2.5 mg/mL of the D101N mutant. After incubation at 37° C. for 3 hours, 8 μL of each reaction was spotted on a Polygram SIL-G TLC plate (Machevey-Nagel, 20×20). The plate was developed in a pre equilibrated chamber with 80:20 ethanol:2.5 M ammonium acetate. After chromatography, the plate was air dried and exposed to a phosphorimaging screen overnight. The reaction using 2.5 mg/ml of wild type enzyme and ¹⁴C-nicotinic acid showed no detectable formation of NAAD.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Another embodiment is a compound of any of the formulae herein made by a process delineated herein, including the processes exemplified in the schemes and examples herein. Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a diseases disorder or symptom thereof delineated herein.

1. A method for identifying a compound which modulates the activity of a Sir2 enzyme, the method comprising:
a) contacting a Sir2 enzyme with a compound under conditions suitable for modulation of the activity of the Sir2 enzyme; and
b) detecting modulation of the activity of the Sir2 enzyme by the compound;
wherein the compound is capable of interacting with the C-pocket of the Sir2 enzymes.

2. A method for identifying a compound which modulates the binding of a Sir2 enzyme, the method comprising:
a) contacting a Sir2 enzyme with a compound under conditions suitable for modulation of the binding of the Sir2 enzyme; and
b) detecting modulation of the binding of the Sir2 enzyme by the compound;
wherein the compound is capable of interacting with the C-pocket of the Sir2 enzymes.

3. A method of modulating the Sir2 activity in a subject, the method comprising administering to the subject a compound identified by:
a) contacting a Sir2 enzyme with a compound under conditions suitable for modulation of the activity of the Sir2 enzyme; and
b) detecting modulation of the activity of the Sir2 enzyme by the compound;
wherein the compound is capable of interacting with the C-pocket of the Sir2 enzymes.

4. A method for identifying a compound which modulates the activity of a Sir2 enzyme, the method comprising:
a) contacting a Sir2 enzyme with a compound under conditions suitable for modulation of the activity of the Sir2 enzyme; and
b) detecting modulation of the activity of the Sir2 enzyme by the compound;
wherein the compound interacts with the flexible loop of the Sir2 enzyme.

5. A method for identifying a compound which modulates the binding of Sir2 enzymes, the method comprising:
a) contacting a Sir2 enzyme with a compound under conditions suitable for modulation of the binding of the Sir2 enzyme; and
b) detecting modulation of the binding of the Sir2 enzyme by the compound;
wherein the compound is interacts with the flexible loop of the Sir2 enzyme.

6. A method of modulating the Sir2 activity in a subject, the method comprising administering to the subject a compound identified by:
a) contacting a Sir2 enzyme with a compound under conditions suitable for binding or modulation of the activity of the Sir2 enzyme; and
b) detecting modulation of the activity or the binding of the Sir2 enzyme by the compound;
wherein the compound interacts with the flexible loop of the Sir2 enzyme.

7. The method of claim 1, wherein the interaction of the of the compound with the Sir2 enzyme is a binding interaction.

8. The method of claim 7, wherein the binding interaction is ionic, covalent, or a non-direct interaction.

9. The method of claim 8, wherein the interaction is covalent.

10. The method of claim 4, wherein the interaction of the compound with the flexible loop causes a conformational change of the Sir2 enzyme.

11. The method of claim 10, wherein the flexible loop becomes rigid.

12. The method of claim 8, wherein the compound has a binding interaction with a conserved aspartic acid in the C pocket of the Sir2 enzyme (Asp103).

13. The method of claim 8, wherein the compound has a binding interaction with a conserved isoleucine in the C pocket of the Sir2 enzyme (Ile102).

14. The method of claim 8, wherein the compound has a binding interaction with a highly conserved residues in the C-pocket, selected from Ala 24, Ile 32, Phe 35 or Ile 102.

15. The method of claim 1, further comprising synthesizing compounds identified in steps a) and b).

16. The method of claim 1, wherein said modulation of the activity of the Sir2 enzyme is detected by direct binding of the compound to the Sir2 enzyme.

17. The method of claim 4, wherein said modulation of the activity of the Sir2 enzyme is detected by direct binding of the compound to the Sir2 enzyme

18. The method of claim 4, wherein said modulation of the activity of the Sir2 enzyme is inhibition of the activity of the Sir2 enzyme.

19. The method of claim 4, wherein said modulation of the activity of the Sir2 enzyme is stimulation of the activity of the Sir2 enzyme.

20. The method of claim 4, wherein said modulation of the activity of the Sir2 enzyme is detected by use of an assay for deacetylation activity.

21-39. (canceled)

40. A method of treating a disorder in a subject, comprising administering to said subject in need thereof, an effective amount of a compound identified in claim 1, such that said subject is treated for said disorder.

41. The method of claim 40, wherein the disorder is age related disorders, cancer or genetic blood diseases, silenced tumor suppressor genes, B-cell-derived non-Hodgkin lymphomas, diffuse large B-cell lymphomas, thalassaemias, sickle cell disease, autoimmune diseases, inflammatory diseases, viral infections, diseases that are associated with a decrease in cell death due to hyperactive apoptosis, cell growth, aging, cell apoptosis, DNA-damaging ionizing radiation, ionizing radiation, metabolic diseases, hyperlipidemia, hypercholesterolemia or type 2 diabetes.

42. The method of claim 41, wherein the age-related disorder is slow replicative aging, cataracts, hypermelanosis, osteoporosis, cerebral cortical atrophy, lymphoid depletion, thymic atrophy, diabetes type II, atherosclerosis, heart disease, lordokyphosis, absence of vigor, lymphoid atrophy, dermal thickening and subcutaneous adipose tissue, atrophy of intestinal villi, skin ulceration, amyloid deposits, and joint diseases.

43-49. (canceled)
